(19) United States
(12) Patent Application Publication
(10) Pub. No.: US 2015/0357580 A1
(43) Pub. Date: Dec. 10, 2015

(54) CARBAZOLE DERIVATIVE AND ORGANIC ELECTROLUMINESCENT DEVICE

(71) Applicant: SAMSUNG DISPLAY CO., LTD., Yongin-City (KR)

(72) Inventor: Hiroaki ITOI, Yokohama (JP)

(21) Appl. No.: 14/725,331

(22) Filed: May 29, 2015

(30) Foreign Application Priority Data
Jun. 6, 2014 (JP) 2014-117512

(51) Int. Cl. H01L 51/00; C07F 7/30; C07F 7/08; C07D 405/14; C07D 409/14

(52) U.S. Cl. CPC H01L 51/0072; C07D 405/14; H01L 51/0073; C07D 409/14; H01L 51/0074; C07F 7/0816; H01L 51/0094; C07F 7/30; H01L 51/0052; H01L 51/0058; H01L 51/5012

(57) ABSTRACT

A carbazole derivative is represented by the following Formula (1):

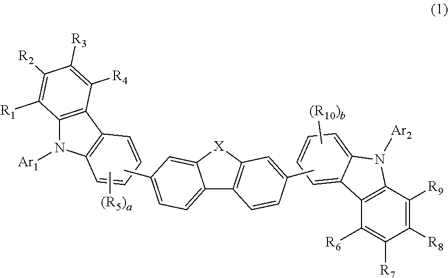

In the above Formula (1), substituted positions of a dibenzoheterole ring in two carbazole rings are different from each other, and X, R₁ to R₁₄, Ar₁ and Ar₂, and a and b are as defined in the specification.

### CROSS-REFERENCE TO RELATED APPLICATION

Japanese Patent Application No. 2014-117512, filed on Jun. 6, 2014, in the Japanese Property Office, and entitled: “Carbazole Derivative and Organic Electroluminescent Device,” is incorporated by reference herein in its entirety.

### BACKGROUND

1. Field

Embodiments relate to a carbazole derivative and an organic electroluminescent device.

2. Description of the Related Art

Recently, an organic electroluminescent device (hereinafter, will be referred to as an organic EL display), which is a self-emitting type device, has been actively developed as an image display apparatus. The organic EL device embodies display through light emission of a light emitting material by recombining holes and electrons injected from an anode and a cathode in an emission layer.

A general organic EL device includes an emission layer and another layer, such as a hole transport layer and an electron transport layer for transporting holes or electrons as carriers to the emission layer, laminated on the emission layer.

### SUMMARY

Embodiments are directed to a carbazole derivative represented by the following Formula (1):

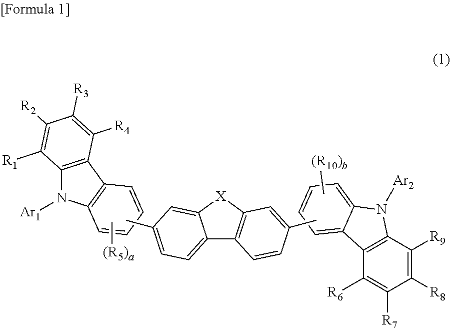

In the above Formula (1), substituted positions of a dibenzoheterole ring in two carbazole rings are different from each other, X is O, S, SiR₁₁R₁₂, or GeR₁₃R₁₄, R₁ to R₁₄ are independently a substituent selected from hydrogen, deuterium, halogen, a substituted or unsubstituted silyl group, a substituted or unsubstituted C₁ to C₁₅ alkyl group, a substituted or unsubstituted C₆ to C₃₀ aryl group and a substituted or unsubstituted C₁ to C₃₀ heteroaryl group, or a substituted or unsubstituted aryl group or heteroaryl group formed by condensing at least two optional and adjacent R₁ to R₁₄, Ar₁ and Ar₂ are independently a substituent selected from a substituted or unsubstituted C₁ to C₁₅ alkyl group, a substituted or unsubstituted C₆ to C₃₀ aryl group, and a substituted or unsubstituted C₁ to C₃₀ heteroaryl group, and a and b are independently an integer from 0 to 3.

X may be O, S or SiR₁₁R₁₂.

The substituted position of the dibenzoheterole ring may be independently position 1, 3, 4, 5, 6 or 8 of the two carbazole rings.

R₁ to R₁₄, Ar₁ and Ar₂ may be independently a substituent selected from hydrogen, deuterium, a phenyl group and a naphthyl group.

An energy level difference of a triplet excited state (T₁) and a ground state (Sₒ) of the carbazole derivative may be from about 2.4 eV to about 3.2 eV.

Embodiments are also directed to an organic electroluminescent (EL) device including a carbazole derivative in at least one organic layer between an anode and an emission layer, or in the emission layer. The carbazole derivative is represented by the following Formula (1):

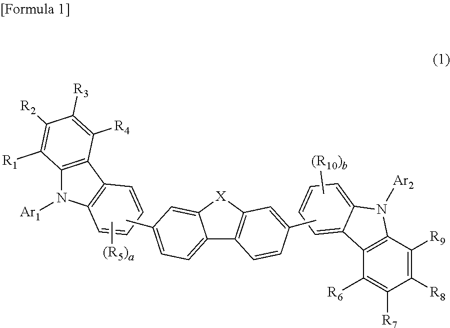

In the above Formula (1), substituted positions of a dibenzoheterole ring in two carbazole rings are different from each other, X is O, S, SiR₁₁R₁₂, or GeR₁₃R₁₄, R₁ to R₁₄ are independently a substituent selected from hydrogen, deuterium, halogen, a substituted or unsubstituted silyl group, a substituted or unsubstituted C₁ to C₁₅ alkyl group, a substituted or unsubstituted C₆ to C₃₀ aryl group and a substituted or unsubstituted C₁ to C₃₀ heteroaryl group, or a substituted or unsubstituted aryl group or heteroaryl group formed by condensing at least two optional and adjacent R₁ to R₁₄, Ar₁ and Ar₂ are independently a substituent selected from a substituted or unsubstituted C₁ to C₁₅ alkyl group, a substituted or unsubstituted C₆ to C₃₀ aryl group, and a substituted or unsubstituted C₁ to C₃₀ heteroaryl group, and a and b are independently an integer from 0 to 3.

X may be O, S or SiR₁₁R₁₂.

The substituted position of the dibenzoheterole ring may be independently position 1, 3, 4, 5, 6 or 8 of each of the two carbazole rings.

R₁ to R₁₄, Ar₁ and Ar₂ may be independently selected from hydrogen, deuterium, a phenyl group and a naphthyl group.

An energy level difference of a triplet excited state (T₁) and a ground state (Sₒ) of the carbazole derivative may be from about 2.4 eV to about 3.2 eV.

A thickness of a layer including the carbazole derivative may be from about 3 nm to about 30 nm.

The carbazole derivative may include at least one of Compounds 1 to 36 (reproduced in the Detailed Description below).

The emission layer may include a condensed polycyclic aromatic compound.

The condensed polycyclic aromatic compound may be at least one compound selected from an anthracene derivative, a pyrene derivative, a fluoranthene derivative, a chrysene derivative, a benzanthracene derivative and a triphenylene derivative.

The condensed polycyclic aromatic compound may be at least one compound selected from a pyrene derivative and an anthracene derivative, the anthracene derivative being represented by the following Formula (2):

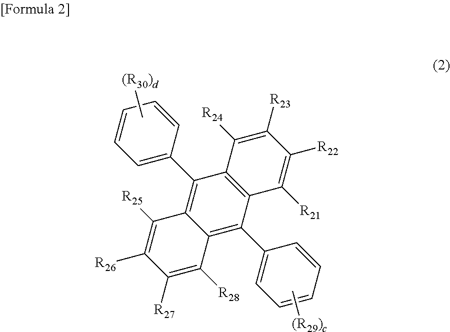

In the above Formula (2), R₂₁ to R₃₀ are independently a substituent selected from hydrogen, deuterium, halogen, a substituted or unsubstituted silyl group, a substituted or unsubstituted C₁ to C₁₅ alkyl group, a substituted or unsubstituted C₆ to C₃₀ aryl group and a substituted or unsubstituted C₁ to C₃₀ heteroaryl group, or a substituted or unsubstituted aryl group or heteroaryl group formed by condensing at least two optional and adjacent R₂₁ to R₃₀, and c and d are independently an integer from 0 to 5.

The condensed polycyclic aromatic compound may include at least one of compounds a-1 to 1-12 (reproduced in the Detailed Description below).

### BRIEF DESCRIPTION OF THE DRAWINGS

Features will become apparent to those of skill in the art by describing in detail exemplary embodiments with reference to the attached drawings in which:

FIG. 1 illustrates a schematic diagram depicting an organic EL device according to an embodiment;

FIG. 2 illustrates a schematic diagram depicting an embodiment of an organic EL device manufactured by using a carbazole derivative according to an embodiment; and

FIG. 3 illustrates a schematic diagram depicting another embodiment of an organic EL device manufactured by using a carbazole derivative according to an embodiment.

### DETAILED DESCRIPTION

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing figures, the dimensions of layers and regions may be exaggerated for clarity of illustration. It will also be understood that when a layer or element is referred to as being “on” another layer or substrate, it can be directly on the other layer or substrate, or intervening layers may also be present. Further, it will be understood that when a layer is referred to as being “between” two layers, it can be the only layer between the two layers, or one or more intervening layers may also be present. Like reference numerals refer to like elements throughout.

<Carbazole Derivative>

First, a carbazole derivative according to an embodiment will be explained. The carbazole derivative according to an embodiment is a compound that may be used as the host material of an emission layer or as a hole transport material in an organic EL device. The carbazole derivative according to an embodiment may be a compound represented by the following Formula (1).

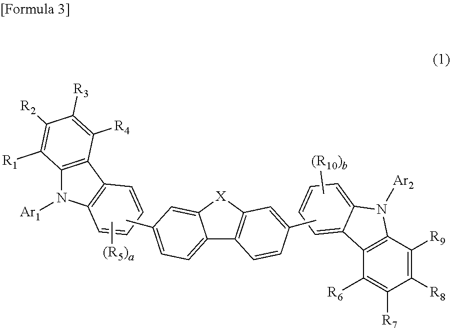

In the above Formula (1),

substituted positions of a dibenzoheterole ring onto two carbazole rings are different from each other,
X is O, S, SiR₁₁R₁₂, or GeR₁₃R₁₄,

R₁ to R₁₄ are independently a substituent selected from the group of hydrogen, deuterium, halogen, a substituted or unsubstituted silyl group, a substituted or unsubstituted C₁ to C₁₅ alkyl group, a substituted or unsubstituted C₆ to C₃₀ aryl group and a substituted or unsubstituted C₁ to C₃₀ heteroaryl group, or a substituted or unsubstituted aryl group or heteroaryl group formed by condensing at least two optional and adjacent R₁ to R₁₄,

Ar₁ and Ar₂ are independently a substituent selected from a substituted or unsubstituted C₁ to C₁₅ alkyl group, a substituted or unsubstituted C₆ to C₃₀ aryl group, and a substituted or unsubstituted C₁ to C₃₀ heteroaryl group, and

a and b are independently an integer from 0 to 3.

The carbazole derivative represented by the above Formula (1) has a structure obtained by connecting two carbazole rings via a dibenzoheterole ring (the term “dibenzoheterole ring” refers to the central portion of Formula (1) that has a similar structure as a carbazole ring, except having X, as further defined, instead of a nitrogen atom). The dibenzoheterole ring may have a high electron tolerance. Accordingly, the carbazole derivative represented by Formula (1) may have improved electron tolerance as compared to a carbazole derivative that does not include the dibenzoheterole ring. The carbazole derivative represented by Formula (1) may improve the emission life of an organic EL device.

The carbazole derivative represented by the above Formula (1) includes highly planar carbazole rings and a dibenzoheterole ring. The carbazole derivative represented by Formula (1) may be highly planar on the whole, and the glass transition temperature (Tg), which is an index of heat resistance, may be increased. Therefore, the carbazole derivative represented by Formula (1) may have high heat resistance, and the emission life of the organic EL device may be improved further. The carbazole derivative represented by Formula (1) may be highly planar on the whole. Accordingly, hole transporting (hopping) efficiency between molecules may be high, and high hole transport capability may be obtained. The carbazole derivative represented by Formula (1) may improve the emission efficiency of the organic EL device.

In the carbazole derivative represented by Formula (1), positions 3 and 7 of the dibenzoheterole ring may be substituted with the carbazole rings. Through the substitution of the carbazole rings at the above positions, the radical cleavage of the carbazole derivative represented by Formula (1) due to a nitrogen atom and a heteroatom may be restrained. The carbazole derivative represented by Formula (1) may have high molecular stability, and the emission life of the organic EL device may be improved further.

The substituted positions of the dibenzoheterole may be different for the two carbazole rings in the carbazole derivative represented by Formula (1). According to the above configuration, the symmetric properties of a molecule in the carbazole derivative represented by Formula (1) may be decreased. Accordingly, crystallinity may be decreased while increasing amorphous properties. Thus, the driving voltage of an organic EL device using the carbazole derivative represented by Formula (1) with high amorphous properties may be decreased further. The carbazole derivative represented by Formula (1) may have high amorphous properties. Accordingly, the carbazole derivative may have better solubility and may be appropriately used in a layer forming method by coating with low cost. The carbazole derivative represented by Formula (1) may improve the productivity of the organic EL device.

In the above Formula (1), X may be, for example, O, S or SiR₁₁R₁₂. When X is O, S or SiR₁₁R₁₂, the dibenzogermole ring connecting the two carbazole rings may have higher stability than other types of dibenzoheterole rings and a greater improving degree of emission life in an organic EL device.

In the above Formula (1), the substituted position of the dibenzoheterole ring may be, for example one of positions 1, 3 to 6 and 8 of the carbazole ring. As verified in the following examples, when the dibenzoheterole ring is substituted at positions 1, 3 to 6 and 8 of a carbazole ring, the improving degree of the emission life in the organic EL device may be improved.

With respect to the designation of the number of the substituted position of the dibenzoheterole ring in this application, in the case that the dibenzoheterole ring is disposed so that the heteroatom is disposed at the lowermost part, the number is designated from the skeleton atom of a ring positioned at the rightmost position (excluding carbon at a condensed position) clockwise. With respect to the designation of the number of the substituted position of the carbazole ring, in the case that the carbazole ring is disposed so that a nitrogen atom is positioned at the uppermost position, the number is designated from the skeleton atom of a ring positioned at the rightmost position (excluding carbon at a condensed position) clockwise.

In the above Formula (1), R₁ to R₁₄, Ar₁ and Ar₂ may be independently an optional substituent. The carbazole derivative represented by Formula (1) may have a large molecular weight because two carbazole rings and one dibenzoheterole ring are included. In the carbazole derivative represented by Formula (1), R₁ to R₁₄, Ar₁ and Ar₂ may preferably be substituted with a small substituent having a relatively low molecular weight so as not to attain an excessively increased molecular weight. For example, R₁ to R₁₄, Ar₁ and Ar₂ may independently and preferably be substituted with a substituent having a relatively small molecular weight selected from the group of hydrogen, deuterium, a phenyl group and a naphthyl group.

The structure of Compounds 1 to 36, as examples of the carbazole derivative according to an embodiment, are illustrated below:

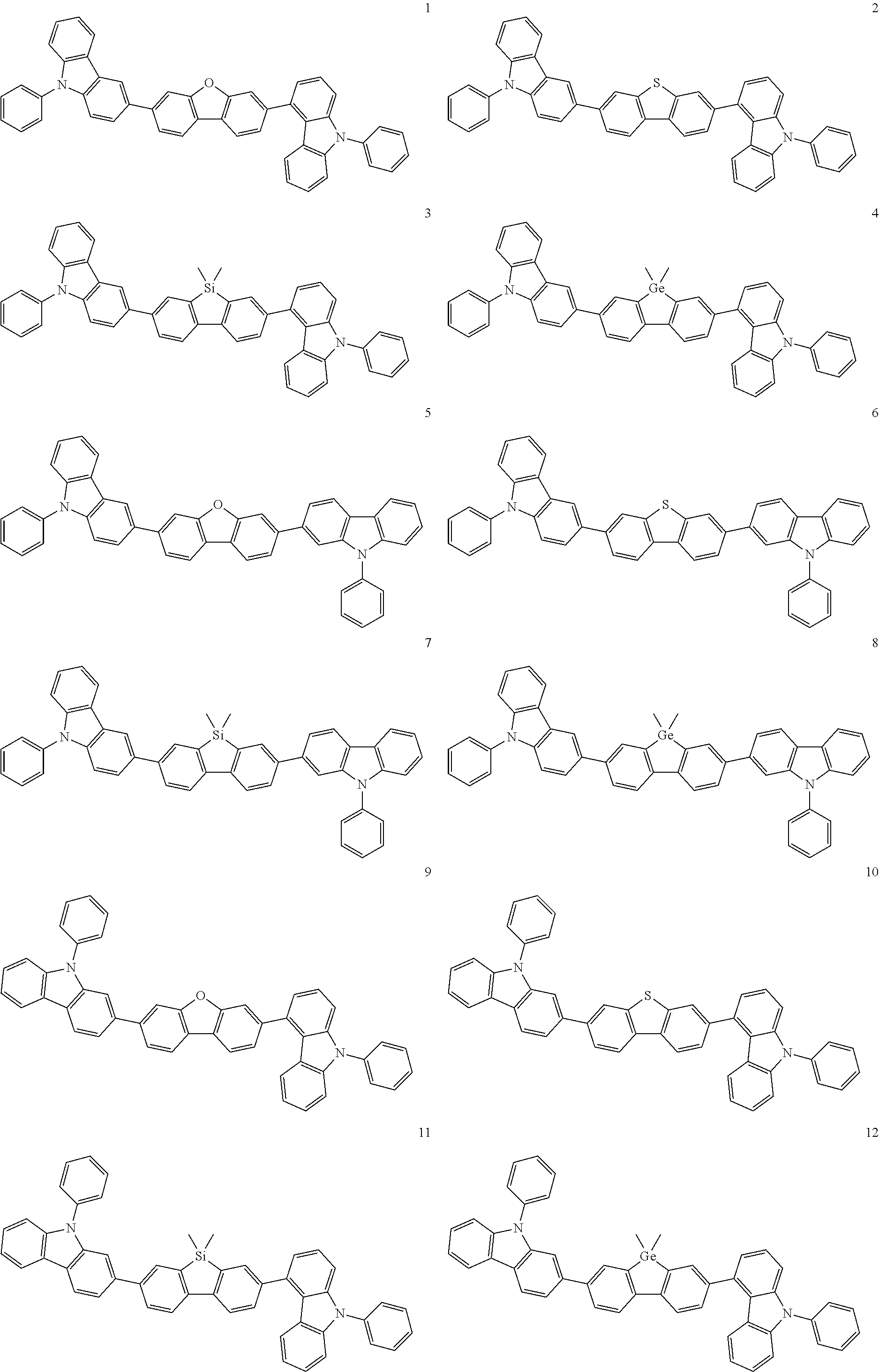

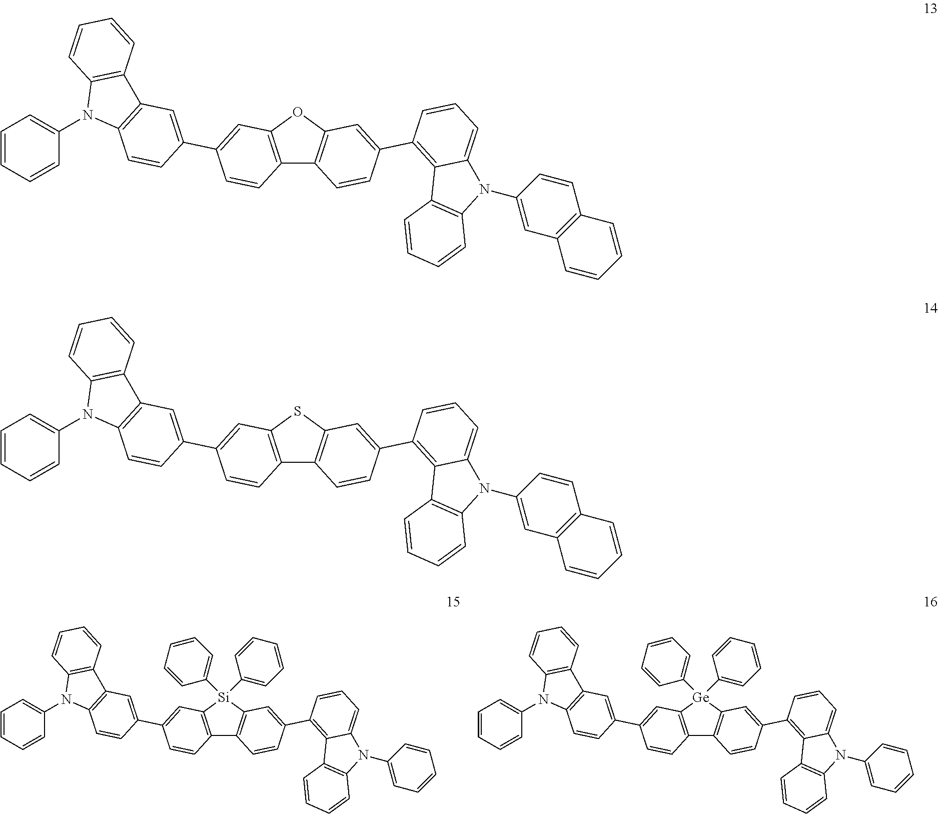

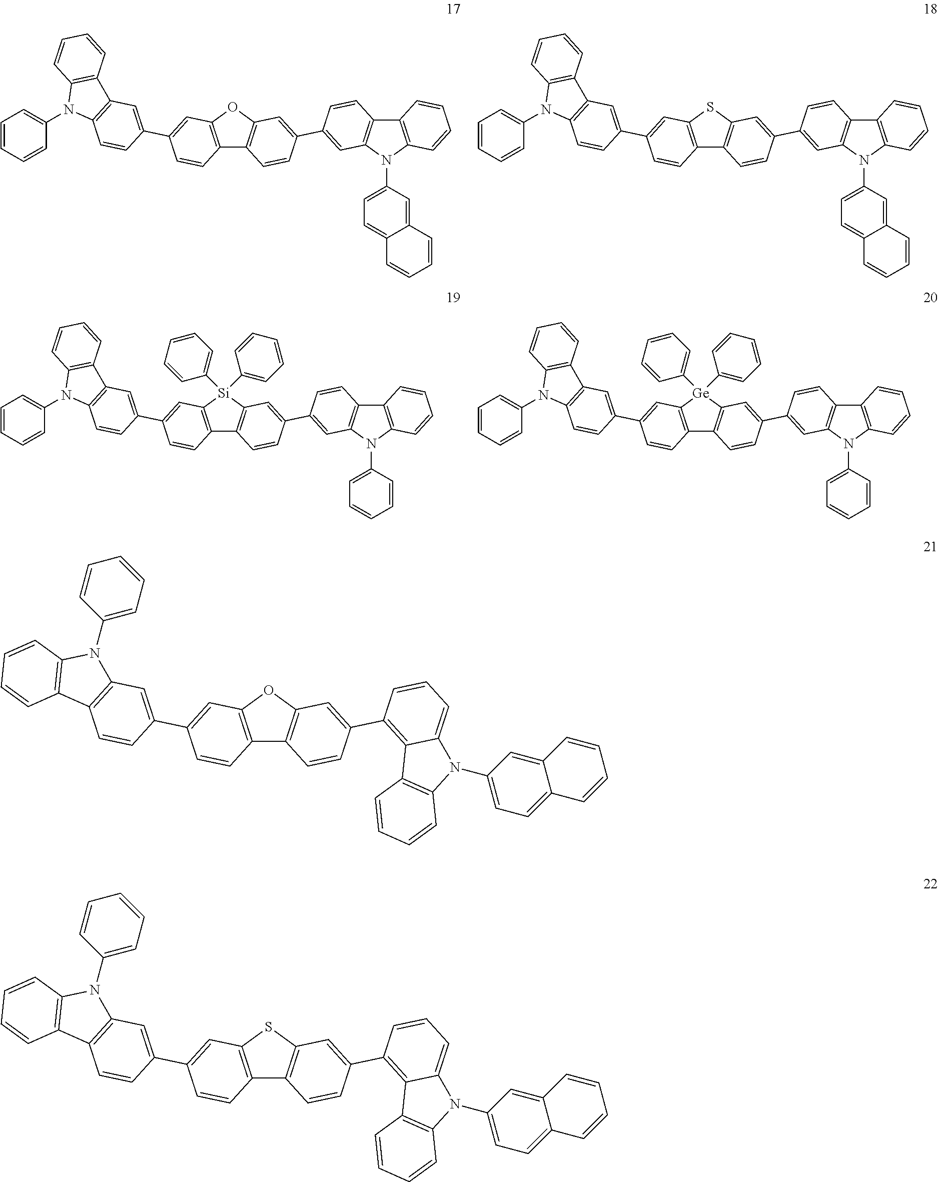

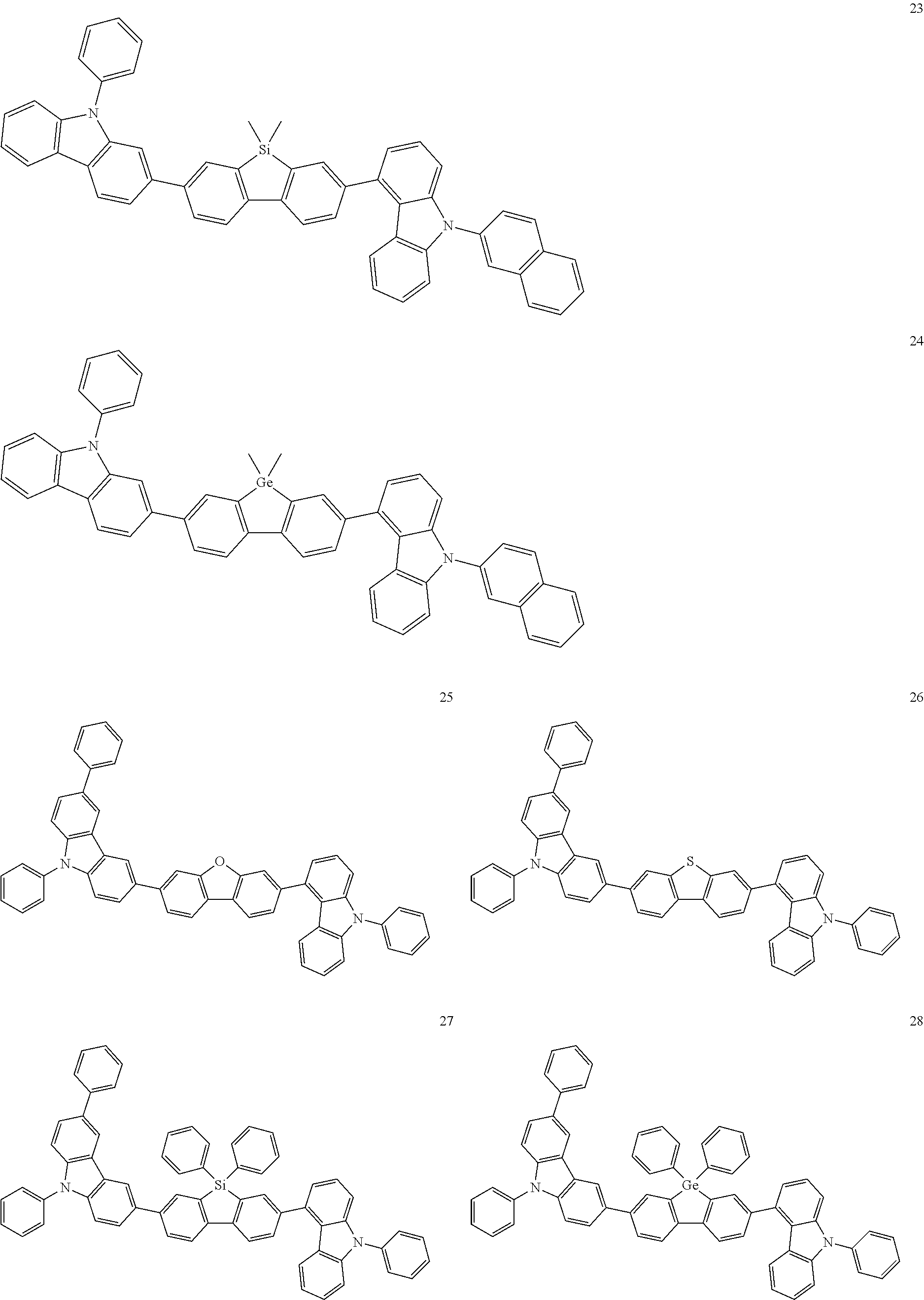

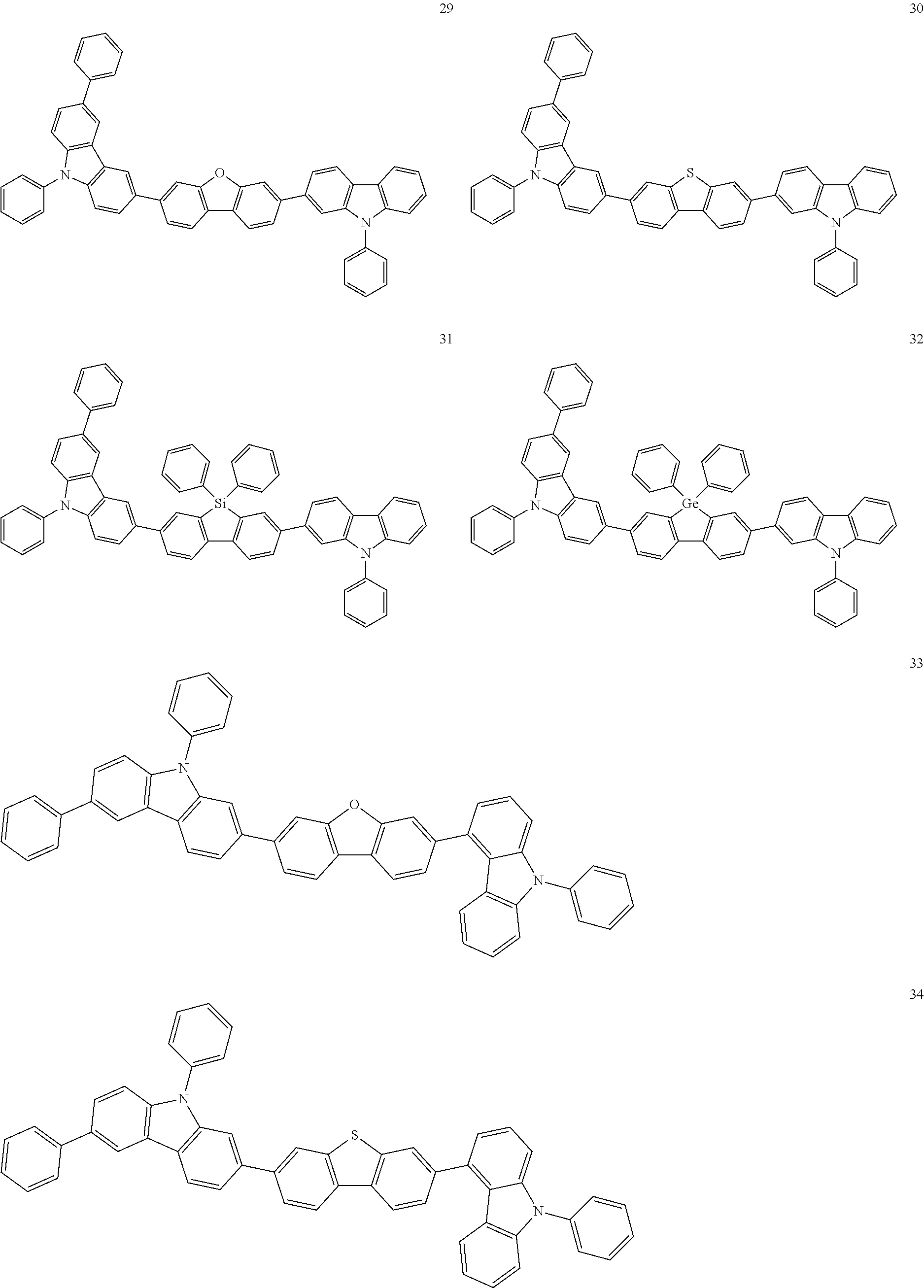

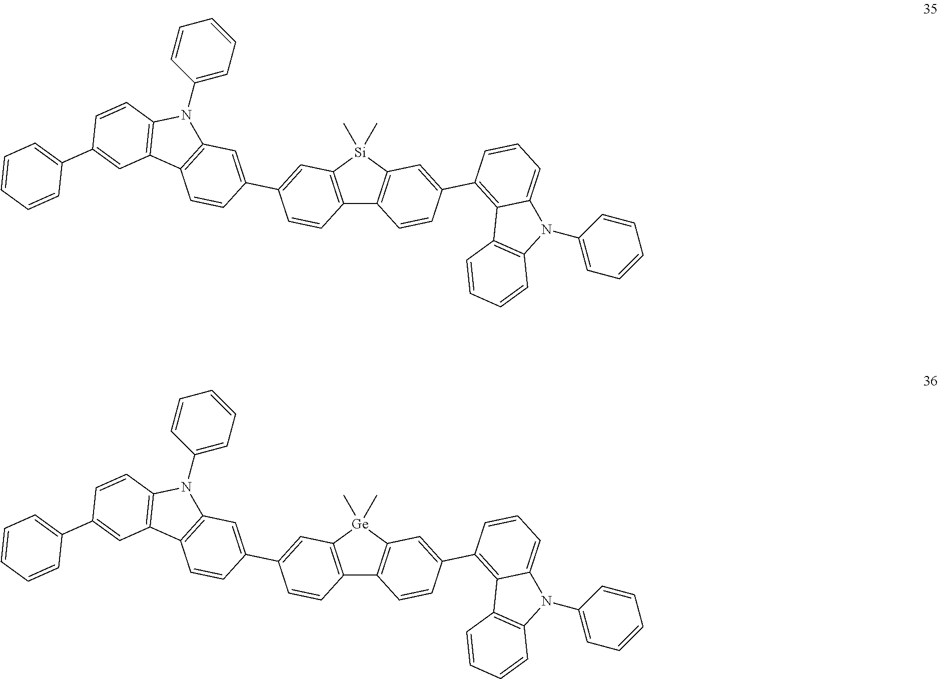

As described above, the carbazole derivative according to an embodiment was explained in detail.

The carbazole derivative according to an embodiment may be appropriately used, for example, as a host material in an emission layer. As verified in the following examples, when the carbazole derivative according to an embodiment is used as the host material in the emission layer, a driving voltage may be decreased, and emission efficiency and emission life may be increased in the organic EL device.

In the case that the carbazole derivative according to an embodiment is used as a host material in an emission layer, the difference of the energy levels between the triplet excited state (T₁) and the ground state (S₀) of the carbazole derivative may be from about 2.4 eV to about 3.2 eV. When the triplet excited state (T₁) has energy level in the above-described range, the carbazole derivative according to an embodiment may effectively move excited energy with respect to a phosphorescent dopant. For example, the carbazole derivative according to an embodiment may be appropriately used as a host material with respect to a green phosphorescent dopant, thereby improving the emission efficiency of an organic EL device.

In addition, the carbazole derivative according to an embodiment may be included in at least one layer (for example, a hole transport layer) disposed between the emission layer and the anode of an organic EL device, and may be appropriately used as a hole transport material. For example, the carbazole derivative according to an embodiment may be included in a hole injection layer or a hole transport layer as a hole transport material. As verified in the following examples, when the carbazole derivative according to an embodiment is used as the hole transport material, the driving voltage of the organic EL device may be decreased, and the emission efficiency and the emission life thereof may be improved.

For example, the carbazole derivative according to an embodiment may be appropriately used as a hole transport material in blue emitting or green emitting organic EL devices, thereby decreasing the driving voltage of the organic EL device and improving the emission efficiency thereof.

When the carbazole derivative according to an embodiment is used as the hole transport material, the thickness of a layer including the carbazole derivative may be, for example, from about 3 nm to about 30 nm. When the thickness of the layer including the carbazole derivative according to an embodiment is greater than about 3 nm, the carbazole derivative according to an embodiment may sufficiently exhibit hole transport capability. When the thickness of the layer including the carbazole derivative according to an embodiment is less than about 30 nm, the layer thickness of the whole organic EL device may small, and the driving voltage thereof may be lower.

When the carbazole derivative according to an embodiment is used as the hole transport material, the emission layer may include a condensed polycyclic aromatic compound. The emission layer may include at least one condensed polycyclic aromatic compound selected from the group of an anthracene derivative, a pyrene derivative, a fluoranthene derivative, a chrysene derivative, a benzanthracene derivative and a triphenylene derivative. For example, the emission layer may include at least one condensed polycyclic aromatic compound selected from the group of an pyrene derivative and an anthracene derivative represented by the following Formula (2). The condensed polycyclic aromatic compound may be included as a host material or as a dopant material in an emission layer.

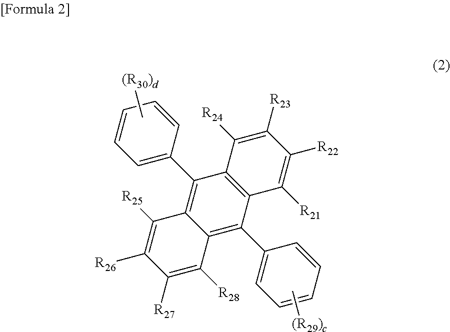

In the above Formula (2),

R₂₁ to R₃₀ are independently a substituent selected from the group of hydrogen, deuterium, halogen, a substituted or unsubstituted silyl group, a substituted or unsubstituted C₁ to C₁₅ alkyl group, a substituted or unsubstituted C₆ to C₃₀ aryl group and a substituted or unsubstituted C₁ to C₃₀ heteroaryl group, or a substituted or unsubstituted aryl group or heteroaryl group formed by condensing at least two optional and adjacent R₂₁ to R₃₀, and

c and d are independently an integer from 0 to 5.

The above-described condensed polycyclic aromatic compound may be a compound functioning as a dopant material or a host material in a blue or green emitting emission layer. As described above, the carbazole derivative according to an embodiment may be appropriately used with respect to the blue or green emitting emission layer. The emission layer including the condensed polycyclic aromatic compound may receive holes from the carbazole derivative according to an embodiment with high efficiency. When the emission layer includes the condensed polycyclic aromatic compound, the driving voltage of the organic EL device according to an embodiment may be decreased further, and the emission efficiency thereof may be improved.

When the carbazole derivative according to an embodiment is used as the hole transport material, examples of the condensed polycyclic aromatic compound included in the emission layer may include the following Compounds a-1 to a-12.

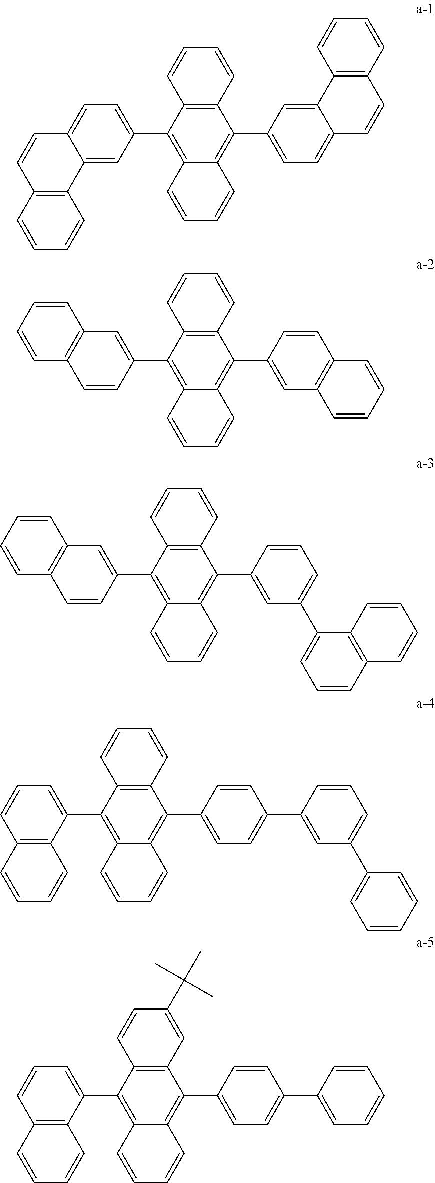

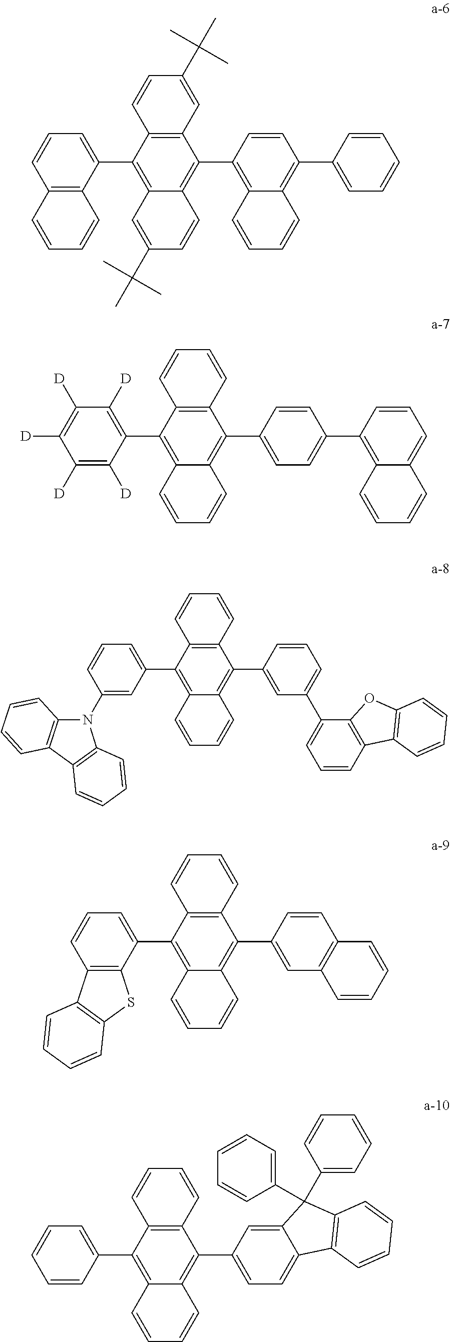

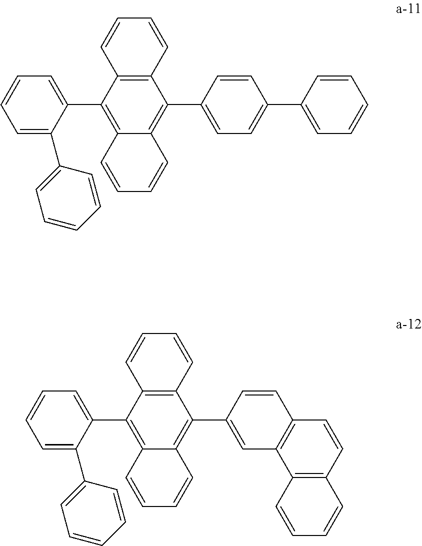

As described above, the carbazole derivative according to an embodiment may have high electron tolerance, and the dibenzoheterole ring thereof may be substituted at positions 3 and 7 with the carbazole rings, thereby restraining reaction in a molecule such as radical cleavage. The life of the organic EL device may be increased due to the carbazole derivative according to an embodiment.

In the carbazole derivative according to an embodiment, the substituted positions of the dibenzoheterole ring in the two carbazole rings are different. Thus, the carbazole derivative according to an embodiment has decreased symmetric properties and improved amorphous properties, thereby decreasing the driving voltage of the organic EL device.

The term “C₁ to C₁₅ alkyl group” refers to a monovalent group of a saturated hydrocarbon having a linear chain or a branched chain, or a saturated hydrocarbon having a ring. Examples of the C₁ to C₁₅ alkyl group may include a methyl group, an ethyl group, a propyl group, an isopropyl group, a cyclopropyl group, a butyl group, an isobutyl group, a tert-butyl group, a cyclobutyl group, a pentyl group, an isopentyl group, a neopentyl group, a cyclopentyl group, a hexyl group, a cyclohexyl group, a heptyl group, an octyl group, a nonyl group, a decyl group, etc.

The term “C₆ to C₃₀ aryl group” refers to a monovalent group including at least one aromatic ring and having 6 to 30 ring carbon atoms. Examples of the C₆ to C₃₀ aryl group may include a phenyl group, a pentalenyl group, an indenyl group, a naphthyl group, an anthracenyl group, an azulenyl group, a heptalenyl group, an acenaphthylenyl group, a phenalenyl group, a fluorenyl group, an anthraquinolyl group, a phenanthryl group, a biphenyl group, a triphenylenyl group, a pyrenyl group, a chrysenyl group, a picenyl group, a perylenyl group, a pentaphenyl group, a pentacenyl group, a tetraphenylene group, a hexaphenyl group, a hexacenyl group, a rubicenyl group, a trinaphthylenyl group, a heptaphenyl group, a pyranthrenyl group, etc.

The term “C₁ to C₃₀ heteroaryl group” refers to a monovalent ring group having at least one aromatic ring including at least one heteroatom selected from N, O, P and S, and C as remainder atoms for forming the ring. Examples of the C₁ to C₃₀ heteroaryl group may include a pyrazolyl group, an imidazolyl group, an oxazolyl group, a thiazolyl group, a triazolyl group, a tetrazolyl group, an oxadiazolyl group, a pyridinyl group, a pyridazinyl group, a pyrimidinyl group, a triazinyl group, a carbazolyl group, an indolyl group, a quinolinyl group, an isoquinolinyl group, a benzoimidazolyl group, an imidazopyridinyl group, an imidazopyrimidinyl group, etc.

The above-described C₁ to C₁₅ alkyl group, C₆ to C₃₀ aryl group and C₁ to C₃₀ heteroaryl group may be substituted with an optional substituent.

<Organic EL Device>

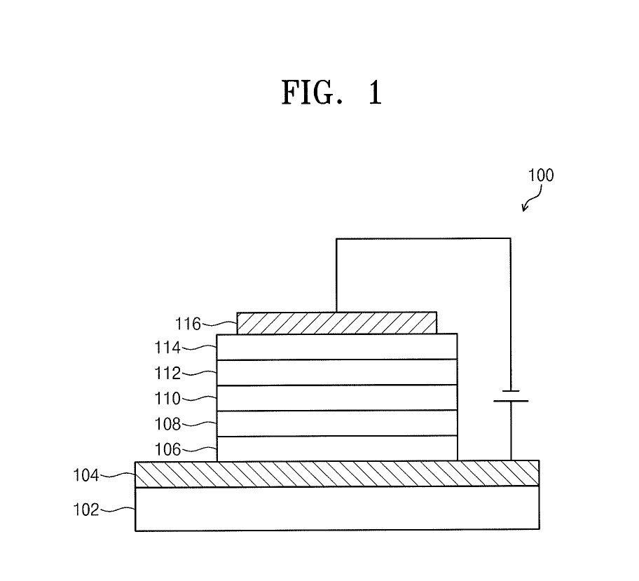

An organic EL device according to an embodiment will be explained referring to FIG. 1. FIG. 1 illustrates a schematic cross-sectional view depicting an embodiment of an organic EL device.

As shown in FIG. 1, an organic EL device 100 according to an embodiment may include a substrate 102, an anode 104 disposed on the substrate 102, a hole injection layer 106 disposed on the anode 104, a hole transport layer 108 disposed on the hole injection layer 106, an emission layer 110 disposed on the hole transport layer 108, an electron transport layer 112 disposed on the emission layer 110, an electron injection layer 114 disposed on the electron transport layer 112 and a cathode 116 disposed on the electron injection layer 114.

The carbazole derivative according to an embodiment may be included in at least one layer positioned between the anode 104 and the emission layer 110. For example, the carbazole derivative according to an embodiment may be included in at least one layer of the hole injection layer 106 and the hole transport layer 108.

The carbazole derivative according to an embodiment may be included as, for example, a host material in the emission layer 110.

The structure of the organic EL device may vary from what is shown in FIG. 1 For example, a portion of layers may be omitted or another layer may be additionally formed in the organic EL device 100 according to an embodiment. In some implementations, each layer of the organic EL device 100 may be formed as a multilayer.

The substrate 102 is a support for laminating each layer of the organic EL device 100. The substrate 102 may be, for example, a transparent glass substrate, a semiconductor substrate formed of silicon (Si), etc., a flexible resin substrate, etc.

The anode 104 may be disposed on the substrate 102. The anode 104 may include, for example, a metal having high work function, an alloy, a conductive compound, etc. For example, the anode 104 may be formed as a transparent electrode by using indium tin oxide (ITO), indium zinc oxide (IZO), tin oxide (SnO₂), zinc oxide (ZnO), etc. In some implementations, the anode 104 may be formed as a reflection type electrode using magnesium (Mg), aluminum (Al), etc.

The hole injection layer 106 may be disposed on the anode 104. The hole injection layer 106 may be a layer that facilitates the easy injection of holes from the anode 104. The hole injection layer 106 may be include, for example, the carbazole derivative according to an embodiment. The hole injection layer 106 may include, for example, 1,4,5,8,9,12-hexaazatriphenylenehexacarbonitrile (HAT(CN)₆), N′N′-diphenyl-N,N′-bis-[4-(phenyl-m-tolyl-amino)-phenyl]-biphenyl-4,4′-diamine (DNTPD), 4,4′,4″-tris(3-methylphenylamino)triphenylamine (m-MTDATA), N.N′-di(1-naphthyl)-N,N′-diphenylbenzidine (NPB), 4,4′,4″-tris{N,N-diamino}triphenylamine (TDATA), 4,4′,4″-tris(N,N-2-naphthylamino)triphenylamine (2-TNATA), polyaniline/dodecylbenzenesulfonic acid (Pani/DBSA), poly(3,4-ethylenedioxy thiophene)/poly(4-styrenesulfonate) (PEDOT/PSS), polyaniline/camphor sulfonic acid (Pani/CSA), polyaniline/poly(4-styrenesulfonate) (PANI/PSS), etc.

The hole transport layer 108 may be disposed on the hole injection layer 106. The hole transport layer 108 may transport holes from the anode 104 to the emission layer 110. The hole transport layer 108 may include, for example, the carbazole derivative according to an embodiment. The hole transport layer 108 may include, for example, N-phenylcarbazole, polyvinyl carbazole, N,N′-bis(3-methylphenyl)-N,N′-diphenyl-[1,1-biphenyl]-4,4′-diamine (TPD), N,N,N′,N′-tetra-(3-methylphenyl)-3,3′-dimethylbenzidine (HMTPD), 4,4′,4″-tris(N-carbazolyl) triphenylamine (TCTA), N,N′-di(1-naphthyl)-N,N′-diphenylbenzidine (NPB), etc.

The emission layer 110 may be disposed on the hole transport layer 108. The emission layer 110 emits light by phosphorescence, fluorescence, etc. The emission layer 110 may include a host material and a dopant material. The dopant material may be one of a luminescent dopant and a phosphorescent dopant.

As the host material of the emission layer 110, the carbazole derivative according to an embodiment may be used. The host material of the emission layer 110 may include, for example, tris(8-quinolinolato)aluminum (Alq3), 4,4′-N,N′-dicarbazole-biphenyl (CBP), poly(n-vinylcarbazole) (PVK), 9,10-di(naphthalene-2-yl)anthracene (ADN), 4,4′,4″-tris(N-carbazolyl)triphenylamine (TCTA), 1,3,5-tris(N-phenyl benzimidazole-2-yl)benzene (TPBI), 3-tert-butyl-9,10-di(naphth-2-yl)anthracene (TBADN), distyrylarylene (DSA), 4,4′-bis(9-carbazole)-2,2′-dimethyl-biphenyl (dmCBP), etc.

The blue dopant material of the emission layer 110 may include, for example, a styryl derivative such as 1,4-bis[2-(3-N-ethylcarbazoryl)vinyl]benzene (BCzVB), 4-(di-p-tolylamino)-4′-[(di-p-tolylamino)styryl]stilbene (DPAVB), N-(4-((E)-2-(6-((E)-4-diphenylamino)styryl)naphthalen-2-yl)vinyl)phenyl]-N-phenylbenzenamine (N-BDAVBi), etc., a perylene derivative such as perylene, 2,5,8,11-tetra-t-butylperylene (TBPe), etc., a pyrene derivative such as pyrene, 1,1-dipyrene, 1,4-dipyrenylbenzene, 1,4-bis(N,N-diphenylamino)pyrene, etc., an iridium (Ir) complex such as bis[2-(4,6-difluorophenyl)pyridinate]picolinate iridium(III) (FIrpic), etc. The green dopant material of the emission layer 110 may include, for example, coumarin or a derivative thereof, an iridium complex such as tris(2-phenylpyridine)iridium(III) (Ir(ppy)₃), etc. The red dopant material of the emission layer 110 may include, for example, rubrene or a derivative thereof, 4-dicyanomethylene-2-(p-dimethylaminostyryl)-6-methyl-4H-pyrane (DCM) and the derivative thereof, an iridium complex such as bis(1-phenylisoquinoline)(acetylacetonate)iridium(III) (Ir(piq)2(acac)), etc., an osmium (Os) complex, a platinum (Pt) complex, etc.

When the carbazole derivative according to an embodiment is included as the host material, the dopant material of the emission layer 110 may be, for example, a green phosphorescent dopant.

When the carbazole derivative according to an embodiment is included in one of the hole injection layer 106 and the hole transport layer 108, the emission layer 110 may include at least one condensed polycyclic aromatic compound selected from the group of an anthracene derivative, a pyrene derivative, a fluoranthene derivative, a chrysene derivative, a benzanthracene derivative and a triphenylene derivative, as the host material or the dopant material. For example, the emission layer 110 may include at least one compound selected from the group of a pyrene derivative and an anthracene derivative represented by the above Formula (2).

The electron transport layer 112 may be disposed on the emission layer 110. The electron transport layer 112 may transport electrons from the cathode 116 to the emission layer 110. The electron transport layer 112 may include, for example, Alq3 or a material having a nitrogen-containing aromatic ring. The material having a nitrogen-containing aromatic ring may be, for example, a material including a pyridine ring, such as 1,3,5-tri[(3-pyridyl)-phen-3-yl]benzene, etc., a material including a triazine ring, such as 2,4,6-tris(3′-(pyridin-3-yl)biphenyl-3-yl)-1,3,5-triazine, etc., and a material including an imidazole derivative, such as 2-(4-(N-phenylbenzimidazolyl-1-ylphenyl)-9,10-dinaphthylanthracene, etc.

The electron injection layer 114 may be disposed on the electron transport layer 112. The electron injection layer may facilitate the easy injection of electrons from the cathode 116. The electron injection layer 114 include, for example, lithium fluoride (LiF), sodium chloride (NaCl), cesium fluoride (CsF), lithium oxide (Li₂O), barium oxide (BaO), etc.

The cathode 116 may be disposed on the electron injection layer 114. The cathode 116 may include, for example, a metal having a low work function, an alloy, a conductive compound, etc. For example, the cathode 116 may include, for example, lithium (Li), magnesium (Mg), aluminum (Al), aluminum-lithium (Al—Li), calcium (Ca), magnesium-indium (Mg—In), magnesium-silver (Mg—Ag), etc. In some implementations, the cathode 116 may be a transparent electrode including ITO, IZO, etc.

Each layer of the organic EL device 100 according to an embodiment may be formed by selecting a suitable material for an organic EL device and an appropriate layer forming method depending on the material used. Examples of layer forming methods include a vacuum deposition method, a sputtering method, various coating methods, etc.

For example, electrode layers such as the anode 104 and the cathode 116 may be formed by a deposition method including an electron beam evaporation method, a hot filament evaporation method or a vacuum deposition method, a sputtering method, a plating method including an electroplating method, or a plating method including an electroless plating method.

In some implementations, the layers including the hole injection layer 106, the hole transport layer 108, the emission layer 110, the electron transport layer 112, the electron injection layer 114, etc. may be formed by a physical vapor deposition method (a PVD method), a printing method such as a screen printing method or an ink jet printing method, a laser transcription method or a coating method such as a spin coat method.

As described above, an embodiment of the organic EL device 100 according to an embodiment was explained.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

### EXAMPLES

[Synthesis of Carbazole Derivative]

First, a synthetic method of a carbazole derivative according to an embodiment will be explained in detail referring to the synthetic methods of the above Compounds 1 to 5 and 9, which are provided for illustration.

(Synthesis of Compound 2)

According to the following Reaction 1, Compound 2 was synthesized as the carbazole derivative according to an embodiment.

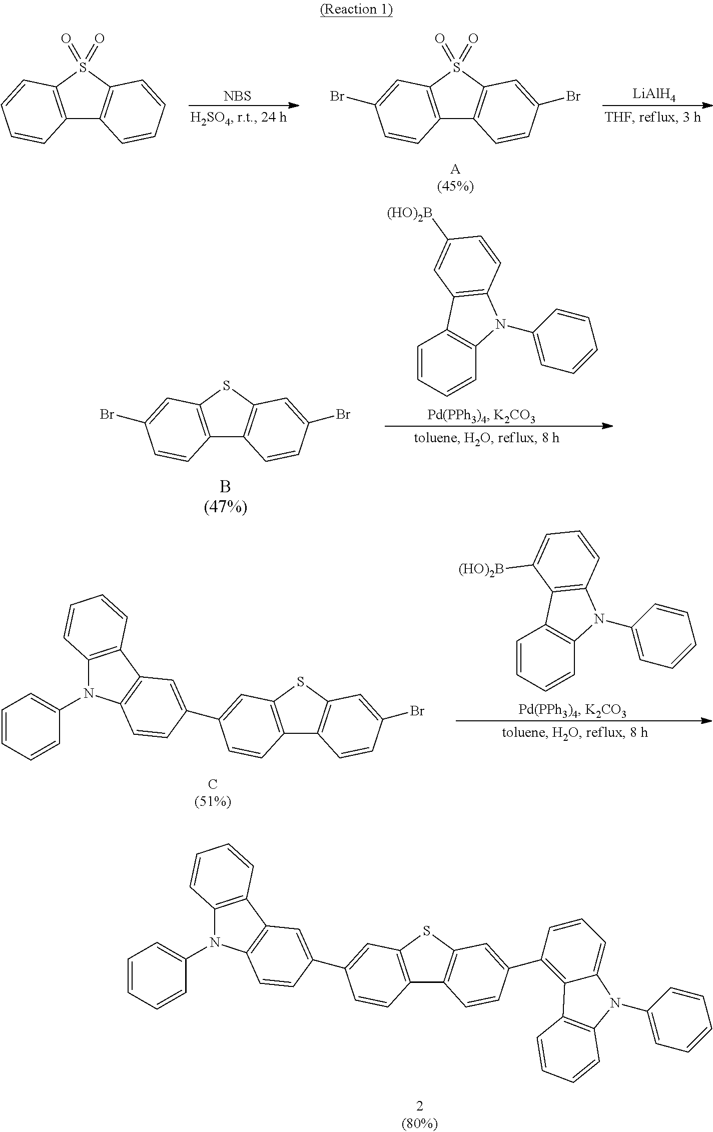

(Synthesis of Compound A)

Under an Ar atmosphere, dibenzo[b,d]thiophene 5,5-dioxide (2.0 g), conc. sulfuric acid (60 mL) and N-bromosuccinimide (NBS, 3.29 g) were added to a 500 mL flask, followed by stirring at room temperature. After stirring, the reaction mixture was poured into cold water, a precipitated solid was filtered with suction, and solvents were distilled. The crude product thus obtained was washed with water and methanol to produce Compound A as a white solid (1.6 g, yield 45%).

¹H-nuclear magnetic resonance (NMR) (DMSO-d₆, 300 MHz) was measured with respect to Compound A. The chemical shift values (δ) of Compound A as measured were 8.33 (d, 2H), 8.11-8.16 (m, 2H), 7.99 (dd, 2H).

(Synthesis of Compound B)

Under an Ar atmosphere, Compound A (10.1 g) and lithium aluminum hydride (LiAlH₄) (1.3 g) were added to a 500 ml flask, followed by heating and refluxing in a tetrahydrofuran (THF) solvent for about 3 hours. After cooling in the air, the reaction mixture was extracted with ethyl acetate, and magnesium sulfate (MgSO₄) and activated clay were added to an extract. After filtering with suction, solvents were distilled. The crude product thus obtained was separated by silica gel column chromatography using a mixture solvent of dichloromethane and hexane to produce Compound B as a pale yellow solid (4.34 g, yield 47%).

Fast atom bombardment mass spectrometry (FAB-MS) was performed with respect to Compound B. The measured molecular weight of Compound B measured was 342.

(Synthesis of Compound C)

Under an Ar atmosphere, Compound B (3.57 g), 9-phenylcarbazole-3-boronic acid (3.01 g), tetrakis(triphenylphosphine)palladium(0) (Pd(PPh₃)₄, 0.33 g) and potassium carbonate (K₂CO₃, 0.77 g) were added to a 300 mL three-necked flask, following by heating while stirring in a toluene solvent (100 mL) at about 90° C. for about 8 hours. After cooling in the air, water was added, an organic layer was separated, and solvents were distilled. The crude product thus produced was separated by silica gel column chromatography using a mixture solvent of dichloromethane and hexane, and recrystallized using a mixture solvent of toluene and hexane to produce Compound C as a while solid (2.70 g, yield 51%).

Here, FAB-MS was performed with respect to Compound C. The measured molecular weight of Compound C measured was 504.

(Synthesis of Compound 2)

Under an Ar atmosphere, Compound C (3.00 g), 9-phenylcarbazole-4-boronic acid (1.59 g), Pd(PPh₃)₄ (0.29 g) and potassium carbonate (0.75 g) were added to a 300 mL three-necked flask, following by heating while stirring in a toluene solvent (90 mL) at about 90° C. for about 8 hours. After cooling in the air, water was added, an organic layer was separated, and solvents were distilled. The crude product thus produced was separated by silica gel column chromatography using a mixture solvent of dichloromethane and hexane, and recrystallized using a mixture solvent of toluene and hexane to produce Compound 2 as a white solid (3.25 g, yield 80%).

FAB-MS was performed with respect to Compound 2. The measured molecular weight of Compound 2 measured was 666. In addition, ¹H-NMR (CDCl₃, 300 MHz) was measured with respect to Compound 2. The measured chemical shift values (δ) of Compound 2 were 8.55 (d, 1H, J=7.82 Hz), 8.12-8.05 (m, 8H), 7.94-7.87 (m, 3H), 7.77 (s, 1H), 7.69-7.58 (m, 4H), 7.63-7.61 (m, 6H), 7.50-7.25 (m, 11H). From the results, the white solid synthesized in Reaction 1 was identified as Compound 2.

(Synthesis of Compound 1)

Compound 1 was synthesized by performing the same procedure in the above Reaction 1 except for using 3,6-dibromodibenzofuran instead of Compound B.

(Synthesis of Compound 3)

Compound 3 was synthesized by performing the same procedure in the above Reaction 1 except for using 2,7-dibromo-9,9-dimethyl-9H-9-silafluorene instead of Compound B.

(Synthesis of Compound 4)

Compound 4 was synthesized by performing the same procedure in the above Reaction 1 except for using 2,7-dibromo-9,9-dimethyl-9H-9-dibenzogermole instead of Compound B.

(Synthesis of Compound 5)

Compound 5 was synthesized by performing the same procedure in the above Reaction 1 except for using 3,6-dibromodibenzofuran instead of dibenzo[b,d]thiophene 5,5-dioxide and using 9-phenylcarbazole-2-boronic acid instead of 9-phenylcarbazole-4-boronic acid.

(Synthesis of Compound 9)

Compound 9 was synthesized by performing the same procedure in the above Reaction 1 except for using 3,6-dibromodibenzofuran instead of dibenzo[b,d]thiophene 5,5-dioxide and using 9-phenylcarbazole-2-boronic acid instead of 9-phenylcarbazole-3-boronic acid.

[Evaluation of Organic EL Device Including Carbazole Derivative as Hole Transport Material]

An organic EL device 200 including the carbazole derivative according to an embodiment as a hole transport material was manufactured by using a vacuum deposition method and the following process and was evaluated.

### Example 1

First, with respect to an ITO-glass substrate patterned and washed in advance, surface treatment was performed using ozone. The layer thickness of an ITO layer on the glass substrate was about 150 nm. Immediately after the ozone treatment, a layer was formed using 2-TNATA as a hole injection material to a layer thickness of about 60 nm on the ITO layer.

Then, a layer was formed using Compound 1 as a hole transport material to a layer thickness of about 30 nm to form a hole transport layer (HTL). In addition, ADN as a host material doped with TBP as a dopant material in an amount of 3 wt % of the total amount of an emission layer was co-deposited to form a layer having a thickness of about 25 nm to form an emission layer.

After that, a layer was formed using Alq3 as an electron transport material to a thickness of about 25 nm. LiF was used as an electron injection material to form a layer having a thickness of about 1 nm, and Al was used to form a layer having a thickness of about 100 nm to form the cathode. The organic EL device 200 was manufactured.

### Examples 2 to 6

An organic EL device was manufactured by performing the same procedure described in Example 1 except for using Compounds 2 to 5 and 9 instead of Compound 1 used in Example 1.

### Comparative Examples 1 to 3

Organic EL devices were manufactured by performing the same procedure described in Example 1 except for using the following Compounds 37 to 39 instead of Compound 1 used in Example 1. Compound 37 was different from the carbazole derivative according to an embodiment in that three carbazole rings were included, and the substituted positions of the carbazole rings were position 2 and position 8 in the dibenzoheterole ring. Compounds 38 and 39 were different from the carbazole derivative according to an embodiment in that the dibenzoheterole ring was not included.

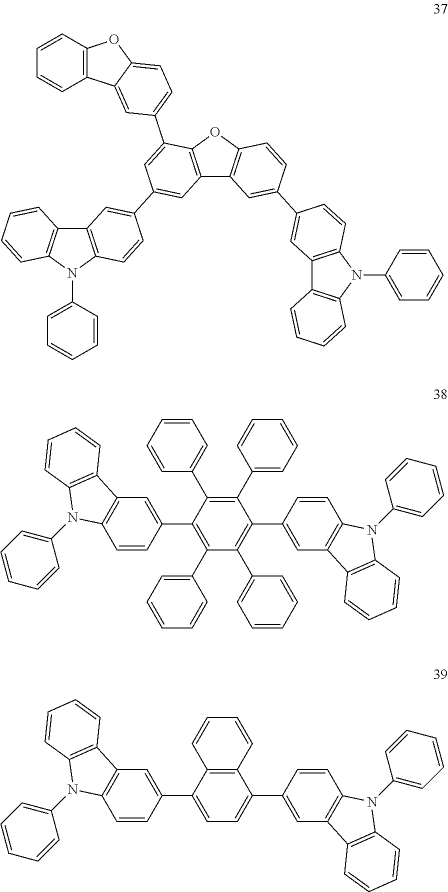 [SMILES: c1ccc(-n2c3ccccc3c3cc(-c4ccc5oc6c(-c7ccc8oc9ccccc9c8c7)cc(-c7ccc8c(c7)c7ccccc7n8-c7ccccc7)cc6c5c4)ccc32)cc1 ; c1ccc(-c2c(-c3ccccc3)c(-c3ccc4c(c3)c3ccccc3n4-c3ccccc3)c(-c3ccccc3)c(-c3ccccc3)c2-c2ccc3c(c2)c2ccccc2n3-c2ccccc2)cc1 ; c1ccc(-n2c3ccccc3c3cc(-c4ccc(-c5ccc6c(c5)c5ccccc5n6-c5ccccc5)c5ccccc45)ccc32)cc1]

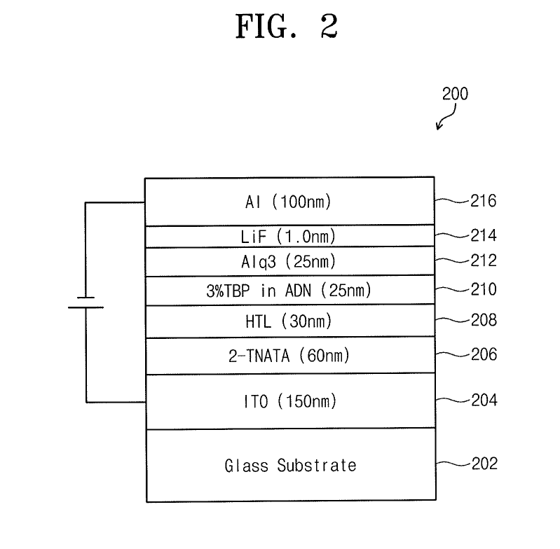

The schematic diagram of the organic EL device according to Examples 1 to 6 and Comparative Examples 1 to 3 is shown in FIG. 2. The organic EL device 200 thus manufactured included a substrate 202, an anode 204 disposed on the substrate 202, a hole injection layer 206 disposed on the anode 204, a hole transport layer 208 disposed on the hole injection layer 206, an emission layer 210 disposed on the hole transport layer 208, an electron transport layer 212 disposed on the emission layer 210, an electron injection layer 214 disposed on the electron transport layer 212, and a cathode 216 disposed on the electron injection layer 214.

The evaluation results of the organic EL devices 200 of Examples 1 to 6 and Comparative Examples 1 to 3 are shown in the following Table 1. For the evaluation of the electroluminescent properties of the organic EL devices 200 thus manufactured, a C9920-11 brightness light distribution characteristics measurement system manufactured by Hamamatsu Photonics Co. was used. In the following Table 1, emission efficiency was measured at current density of about 10 mA/cm² and half life was measured at about 1,000 cd/m².

TABLE 1

| | Hole | Current | Driving | Emission | Emission |
|---|---|---|---|---|---|
| | transport | density | voltage | efficiency | life |
| | material | [mA/cm²] | [V] | [cd/A] | LT₅₀[h] |
| Example 1 | Compound 1 | 10 | 5.2 | 7.9 | 2,000 |
| Example 2 | Compound 2 | 10 | 5.5 | 7.1 | 2,600 |
| Example 3 | Compound 3 | 10 | 5.9 | 6.9 | 2,200 |
| Example 4 | Compound 4 | 10 | 6.3 | 6.1 | 1,800 |
| Example 5 | Compound 5 | 10 | 5.3 | 6.3 | 1,500 |
| Example 6 | Compound 9 | 10 | 5.4 | 6.1 | 1,800 |
| Comparative | Compound | 10 | 6.3 | 6.1 | 1,400 |
| Example 1 | 37 | | | | |
| Comparative | Compound | 10 | 6.5 | 5.2 | 1,000 |
| Example 2 | 38 | | | | |
| Comparative | Compound | 10 | 7.6 | 5.1 | 1,300 |
| Example 3 | 39 | | | | |

Referring to the results of Table 1, it can be seen that the emission life was increased for Examples 1 to 6 using the carbazole derivatives of Compounds 1 to 5 and 9 according to exemplary embodiments as the hole transport materials when compared to those for Comparative Examples 1 to 3.

For example, the symmetric properties of a molecule were broken by introducing a dibenzoheterole ring between two different carbazole rings in the carbazole derivatives used in Examples 1 to 6. Desirable amorphous properties were realized in the organic EL devices. The driving voltage was lowered, and the emission efficiency was improved for Examples 1 to 6, compared to the driving voltage and emission efficiency of Comparative Examples 2 and 3. In addition, when a carbazole derivative including a dibenzoheterole ring having high electron tolerance was used, as in Examples 1 to 6, the emission life was increased as compared to that of Comparative Examples 2 and 3.

The substituted positions of the carbazole rings were at position 3 and position 7 in the dibenzoheterole ring in the carbazole derivative used in Examples 1 to 6. Accordingly, radical cleavage due to a nitrogen atom and a heteroatom could be restrained. The emission life for Examples 1 to 6 was increased when compared to that of Comparative Example 1 using a carbazole derivative in which the substituted positions of the carbazole rings in the dibenzoheterole ring differed from that of the carbazole derivatives according to embodiments.

In the emission layer for Examples 1 to 6, an anthracene derivative (ADN, Compound a-2) was included when the carbazole derivative according to an embodiment was used as the hole transport material. Accordingly the emission life was increased. The thickness of the hole transport layer of Examples 1 to 6 was about 30 nm, which is a suitable range of a layer including the carbazole derivative according to an embodiment.

The carbazole derivative used in Examples 1 to 3 was the compound of Formula (1) where X was O, S or SiR₁₁R₁₂. The stability thereof was very high. The emission life of the carbazole derivatives according to Examples 1 to 3 was longer than Example 4 using the carbazole derivative where X is GeR₁₃R₁₄, and the stability thereof was not as high. In Formula (1), the emission life of Example 2 using the carbazole derivative where X was S and the stability thereof was high, was the longest.

The emission life of Examples 1 to 4 using the carbazole derivative where the dibenzoheterole ring was substituted at position 3 or position 4 of the carbazole rings tended to be longer than that of Examples 5 and 6 using the carbazole derivative where the dibenzoheterole ring was substituted at position 2 of one carbazole ring.

As explained above, the emission life of the organic EL device may be increased by using the carbazole derivative according to an embodiment as the hole transport material.

[Evaluation of Organic EL Device Including Carbazole Derivative as Host Material]

An organic EL device 300 including the carbazole derivative according to an embodiment as the host material of the emission layer was manufactured by a vacuum deposition method, and evaluation thereof was conducted.

### Example 11

First, with respect to an ITO-glass substrate patterned and washed in advance, surface treatment was performed using ozone. The layer thickness of an ITO layer on the glass substrate was about 150 nm. Immediately after the ozone treatment, a layer was formed using 2-TNATA as a hole injection material to a layer thickness of about 60 nm on the ITO layer.

Then, a layer was formed using HMTPD as a hole transport material to a layer thickness of about 30 nm. Compound 1 as a host material doped with Ir(ppy)₃ as a dopant material in an amount of about 20 wt % of the total amount of an emission layer was co-deposited to from a layer to a thickness of about 25 nm to form an emission layer.

After that, a layer was formed using Alq3 as an electron transport material to a thickness of about 25 nm. LiF was used as an electron injection material to form a layer to a thickness of about 1 nm, and Al was used to form a layer to a thickness of about 100 nm to form a cathode. The organic EL device 300 was manufactured.

### Examples 12 to 16

Organic EL devices were manufactured by performing the same procedure described in Example 11 except for using Compounds 2 to 5 and 9 instead of Compound 1 used in Example 11.

### Comparative Examples 11 to 13

Organic EL devices were manufactured by performing the same procedure described in Example 11 except for using Compounds 37 to 39 instead of Compound 1 used in Example 11. Compound 37 was different from the carbazole derivative according to an embodiment in that three carbazole rings were included, and the substituted positions of the carbazole rings were position 2 and position 8 in the dibenzoheterole ring. Compounds 38 and 39 were different from the carbazole derivative according to an embodiment in that the dibenzoheterole ring was not included.

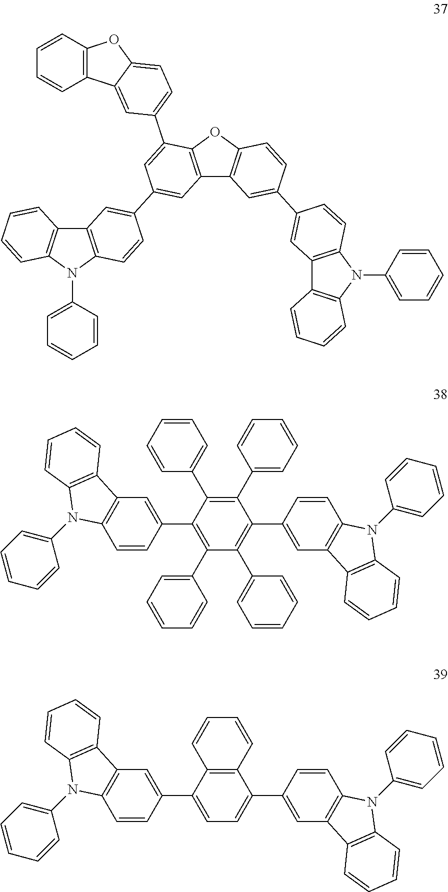 [SMILES: c1ccc(-n2c3ccccc3c3cc(-c4ccc5oc6c(-c7ccc8oc9ccccc9c8c7)cc(-c7ccc8c(c7)c7ccccc7n8-c7ccccc7)cc6c5c4)ccc32)cc1 ; c1ccc(-c2c(-c3ccccc3)c(-c3ccc4c(c3)c3ccccc3n4-c3ccccc3)c(-c3ccccc3)c(-c3ccccc3)c2-c2ccc3c(c2)c2ccccc2n3-c2ccccc2)cc1 ; c1ccc(-n2c3ccccc3c3cc(-c4ccc(-c5ccc6c(c5)c5ccccc5n6-c5ccccc5)c5ccccc45)ccc32)cc1]

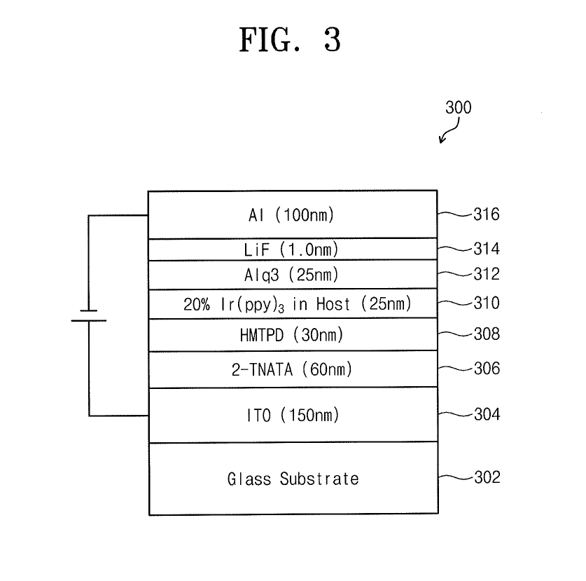

The schematic diagram of the organic EL device according to Examples 11 to 16 and Comparative Examples 11 to 13 is shown in FIG. 3. The organic EL device 300 thus manufactured includes a substrate 302, an anode 304 disposed on the substrate 302, a hole injection layer 306 disposed on the anode 304, a hole transport layer 308 disposed on the hole injection layer 306, an emission layer 310 disposed on the hole transport layer 308, an electron transport layer 312 disposed on the emission layer 310, an electron injection layer 314 disposed on the electron transport layer 312, and a cathode 316 disposed on the electron injection layer 314.

The evaluation results of the organic EL devices 300 of Examples 11 to 16 and Comparative Examples 11 to 13 are shown in the following Table 2. For the evaluation of the electroluminescent properties of the organic EL devices 300 thus manufactured, a C9920-11 brightness light distribution characteristics measurement system manufactured by Hamamatsu Photonics Co. was used. In the following Table 2, emission efficiency was measured at current density of about 10 mA/cm² and half life was measured at about 1,000 cd/m².

TABLE 2

| | | Current | Driving | Emission | Emission |
|---|---|---|---|---|---|
| | Host | density | voltage | efficiency | life |
| | material | [mA/cm²] | [V] | [cd/A] | LT₅₀[h] |
| Example 11 | Compound 1 | 10 | 4.3 | 34.6 | 2,400 |
| Example 12 | Compound 2 | 10 | 4.5 | 34.0 | 2,300 |
| Example 13 | Compound 3 | 10 | 4.7 | 31.9 | 2,000 |
| Example 14 | Compound 4 | 10 | 4.9 | 32.0 | 1,800 |
| Example 15 | Compound 5 | 10 | 4.4 | 34.1 | 1,900 |
| Example 16 | Compound 9 | 10 | 4.6 | 35.0 | 2,200 |
| Comparative | Compound | 10 | 5.2 | 30.1 | 1,400 |
| Example 11 | 37 | | | | |
| Comparative | Compound | 10 | 5.3 | 29.3 | 1,000 |
| Example 12 | 38 | | | | |
| Comparative | Compound | 10 | 5.5 | 28.7 | 1,200 |
| Example 13 | 39 | | | | |

Referring to the results in Table 2, a driving voltage was decreased, emission efficiency was improved and emission life was increased with respect to Examples 11 to 16 using Compounds 1 to 5 and 9, which were the carbazole derivatives according to exemplary embodiments as the host material of the emission layer, as compared to those for Comparative Examples 11 to 13.

For example, the carbazole derivatives used in Examples 11 to 16 introduced the dibenzoheterole ring between two carbazole rings and realized the energy level of a triplet excited state (T₁) preferable as a phosphorescent dopant. Thus, the driving voltage was decreased, and emission efficiency was improved for Examples 11 to 16 as compared to Comparative Examples 12 and 13. In addition, Examples 11 to 16 used the carbazole derivative including the dibenzoheterole ring having high electron tolerance. Accordingly, the emission life thereof was increased when compared with that of Comparative Examples 12 and 13.

In the carbazole derivatives used in Examples 11 to 16, the substituted positions of the carbazole rings in the dibenzoheterole ring were position 3 and position 7. Accordingly, radical cleavage due to a nitrogen atom and a heteroatom could be restrained. Thus, the emission life of Examples 11 to 16 may be increased when compared to that of Comparative Example 11 including the carbazole derivative in which the substituted position of the carbazole ring in the dibenzoheterole ring differed from that of the carbazole derivatives according to embodiments.

The carbazole derivatives used in Examples 11 to 13 were the compound of Formula (1) where X was O, S or SiR₁₁R₁₂, and the stability thereof was very high. Therefore, it may be found that the emission life of Examples 11 to 13 was longer than that of Example 14 using the carbazole derivative where X was GeR₁₃R₁₄, and the stability thereof was not as high.

In addition, the emission life of Examples 11 to 14 using the carbazole derivative where the dibenzoheterole ring was substituted at position 3 or position 4 of the carbazole ring tended to be longer than that of Examples 15 and 16 using the carbazole derivative where the dibenzoheterole ring was substituted at position 2 of one carbazole ring.

As explained above, the emission life of the organic EL device may be increased by using the carbazole derivative according to an embodiment as the host material of the emission layer.

Therefore, the carbazole derivatives according to exemplary embodiments may have a high electron tolerance. When the dibenzoheterole ring is substituted with the carbazole rings at position 3 and position 7, radical cleavage due to a nitrogen atom and a heteroatom may be restrained, thereby realizing the long life of the organic EL device. In the carbazole derivatives according to exemplary embodiments, the substituted positions of the dibenzoheterole ring in the two carbazole rings are different from each other. Accordingly, amorphous properties may be improved, thereby decreasing the driving voltage of the organic EL device.

The carbazole derivative according to an embodiment may be used in other luminescent devices or luminescent apparatuses besides the illustrated organic EL devices. In some implementations, the organic EL devices shown in FIGS. 1 and 3 may be used in an organic EL device of a passive-matrix driving type, or in an organic EL device of an active-matrix driving type.

By way of summation and review, to improve the emission efficiency and the emission life of the organic EL device, various compounds have been examined as materials used in each layer. For example, an aromatic amine compound has been suggested as the material of an organic EL device. A heterocyclic compound substituted with a carbazole ring may be used as the host material of an emission layer. A carbazole derivative substituted with a condensed ring may be used as a hole transport material. However, such compounds may have insufficient electron tolerance, and an organic EL device using the compounds may have a defect of short emission life. Thus, a material compound capable of further improving the emission life of the organic EL device is desirable.

Embodiments provide a novel and improved carbazole derivative having greater stability and that may provide an increased emission life of an organic EL device. Embodiment further provide an organic EL device including the carbazole derivative having a decreased driving voltage and an increased emission life and emission efficiency.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope thereof as set forth in the following claims.

What is claimed is:

1. A carbazole derivative represented by the following Formula (1): 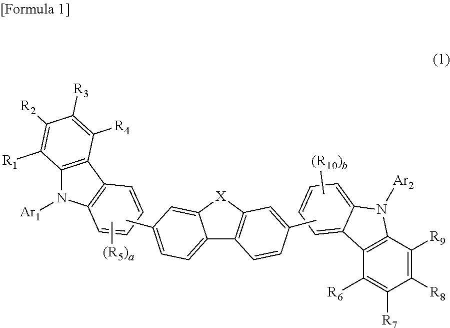
wherein, in the above Formula (1),
substituted positions of a dibenzoheterole ring in two carbazole rings are different from each other,
X is O, S, SiR₁₁R₁₂, or GeR₁₃R₁₄,
R₁ to R₁₄ are independently a substituent selected from hydrogen, deuterium, halogen, a substituted or unsubstituted silyl group, a substituted or unsubstituted C₁ to C₁₅ alkyl group, a substituted or unsubstituted C₆ to C₃₀ aryl group and a substituted or unsubstituted C₁ to C₃₀ heteroaryl group, or a substituted or unsubstituted aryl group or heteroaryl group formed by condensing at least two optional and adjacent R₁ to R₁₄,
Ar₁ and Ar₂ are independently a substituent selected from a substituted or unsubstituted C₁ to C₁₅ alkyl group, a substituted or unsubstituted C₆ to C₃₀ aryl group, and a substituted or unsubstituted C₁ to C₃₀ heteroaryl group, and
a and b are independently an integer from 0 to 3.

2. The carbazole derivative as claimed in claim 1, wherein X is O, S or SiR₁₁R₁₂.

3. The carbazole derivative as claimed in claim 1, wherein the substituted position of the dibenzoheterole ring is independently position 1, 3, 4, 5, 6 or 8 of the two carbazole rings.

4. The carbazole derivative as claimed in claim 1, wherein R₁ to R₁₄, Ar₁ and Ar₂ are independently a substituent selected from hydrogen, deuterium, a phenyl group and a naphthyl group.

5. The carbazole derivative as claimed in claim 1, wherein an energy level difference of a triplet excited state (T₁) and a ground state (S₀) of the carbazole derivative is from about 2.4 eV to about 3.2 eV.

6. An organic electroluminescent (EL) device, comprising a carbazole derivative in at least one organic layer between an anode and an emission layer, or in the emission layer,
wherein the carbazole derivative is represented by the following Formula (1): 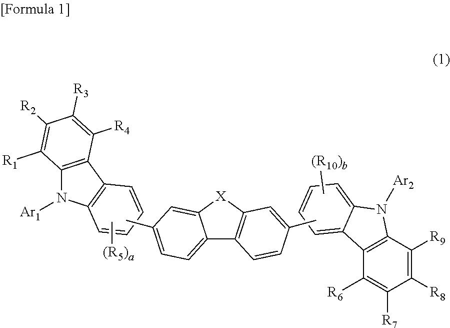
wherein, in the above Formula (1),
substituted positions of a dibenzoheterole ring in two carbazole rings are different from each other,
X is O, S, SiR₁₁R₁₂, or GeR₁₃R₁₄,
R₁ to R₁₄ are independently a substituent selected from hydrogen, deuterium, halogen, a substituted or unsubstituted silyl group, a substituted or unsubstituted C₁ to C₁₅ alkyl group, a substituted or unsubstituted C₆ to C₃₀ aryl group and a substituted or unsubstituted C₁ to C₃₀ heteroaryl group, or a substituted or unsubstituted aryl group or heteroaryl group formed by condensing at least two optional and adjacent R₁ to R₁₄,
Ar₁ and Ar₂ are independently a substituent selected from a substituted or unsubstituted C₁ to C₁₅ alkyl group, a substituted or unsubstituted C₆ to C₃₀ aryl group, and a substituted or unsubstituted C₁ to C₃₀ heteroaryl group, and
a and b are independently an integer from 0 to 3.

7. The organic EL device as claimed in claim 6, wherein X is O, S or SiR₁₁R₁₂.

8. The organic EL device as claimed in claim 6, wherein the substituted position of the dibenzoheterole ring is independently position 1, 3, 4, 5, 6 or 8 of each of the two carbazole rings.

9. The organic EL device as claimed in claim 6, wherein R₁ to R₁₄, Ar₁ and Ar₂ are independently selected from hydrogen, deuterium, a phenyl group and a naphthyl group.

10. The organic EL device as claimed in claim 6, wherein an energy level difference of a triplet excited state (T₁) and a ground state (Sₒ) of the carbazole derivative is from about 2.4 eV to about 3.2 eV.

11. The organic EL device as claimed in claim 6, wherein a thickness of a layer including the carbazole derivative is from about 3 nm to about 30 nm.

12. The organic EL device as claimed in claim 6, wherein the carbazole derivative includes at least one of the following Compounds 1 to 36:

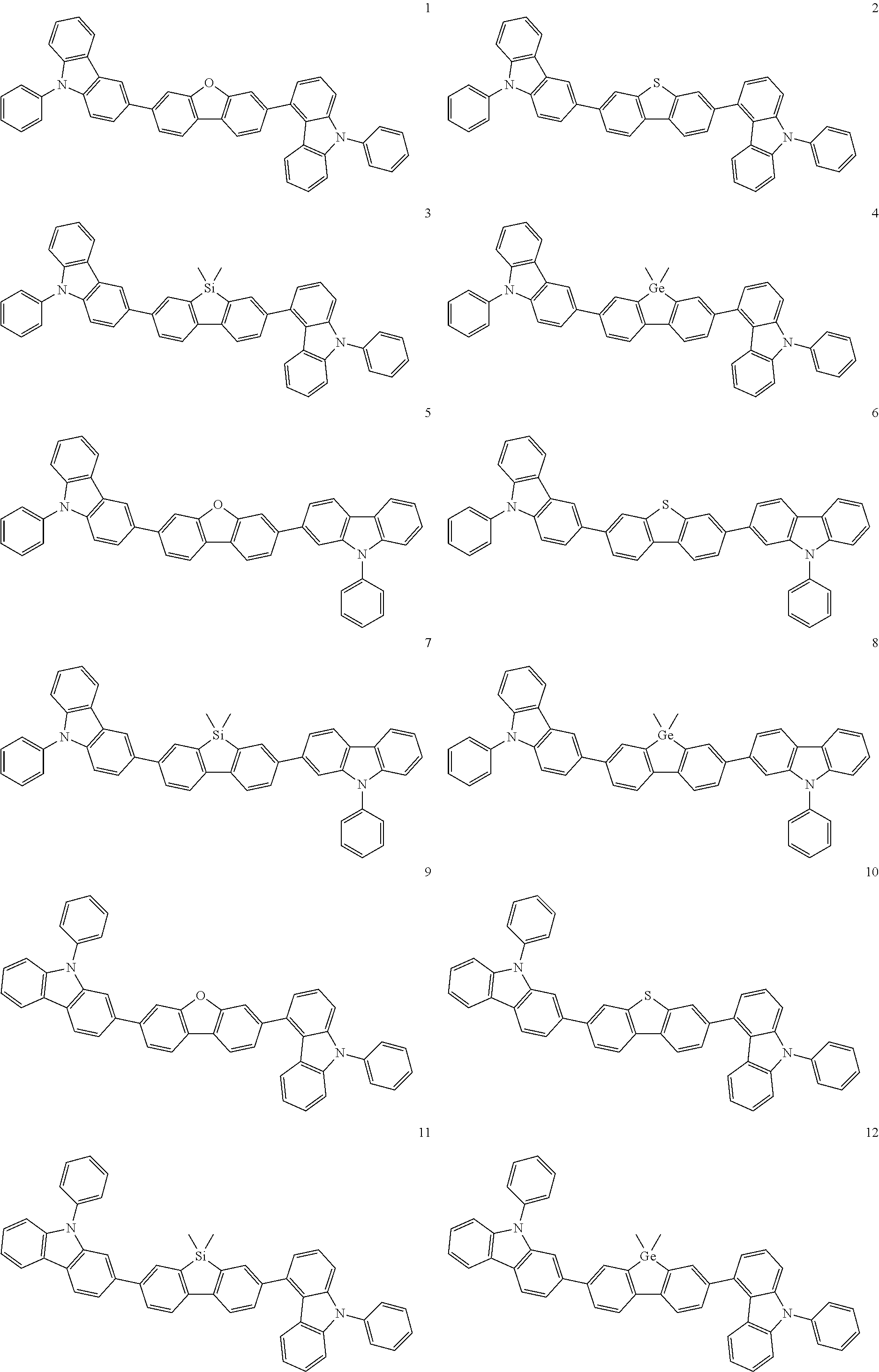

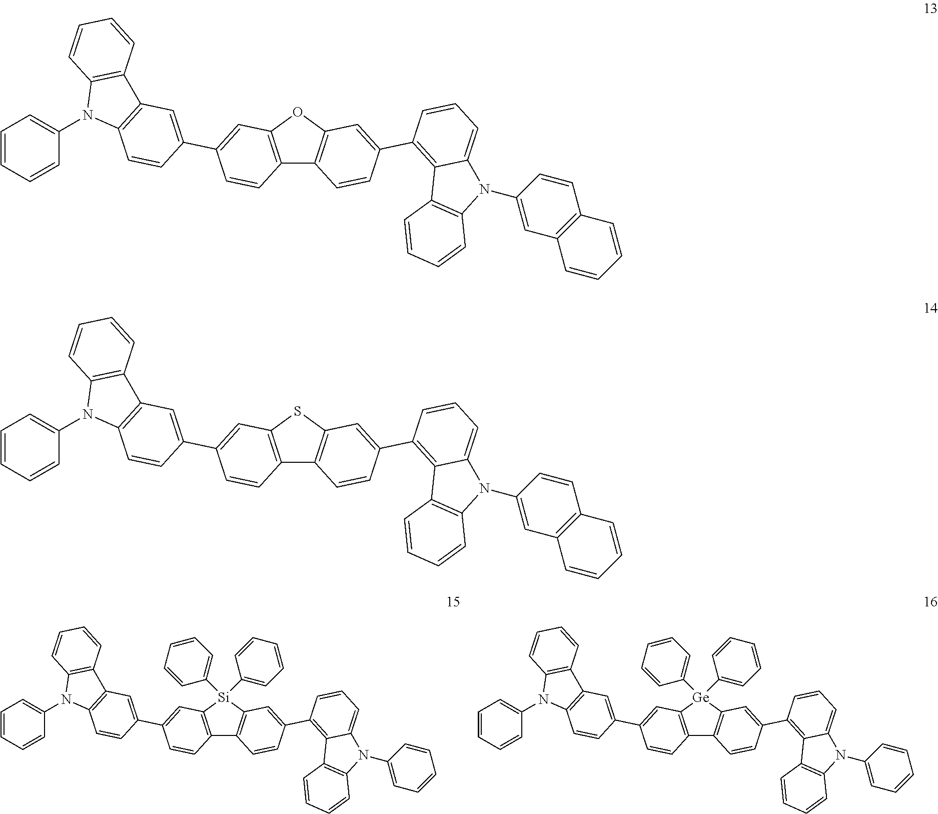

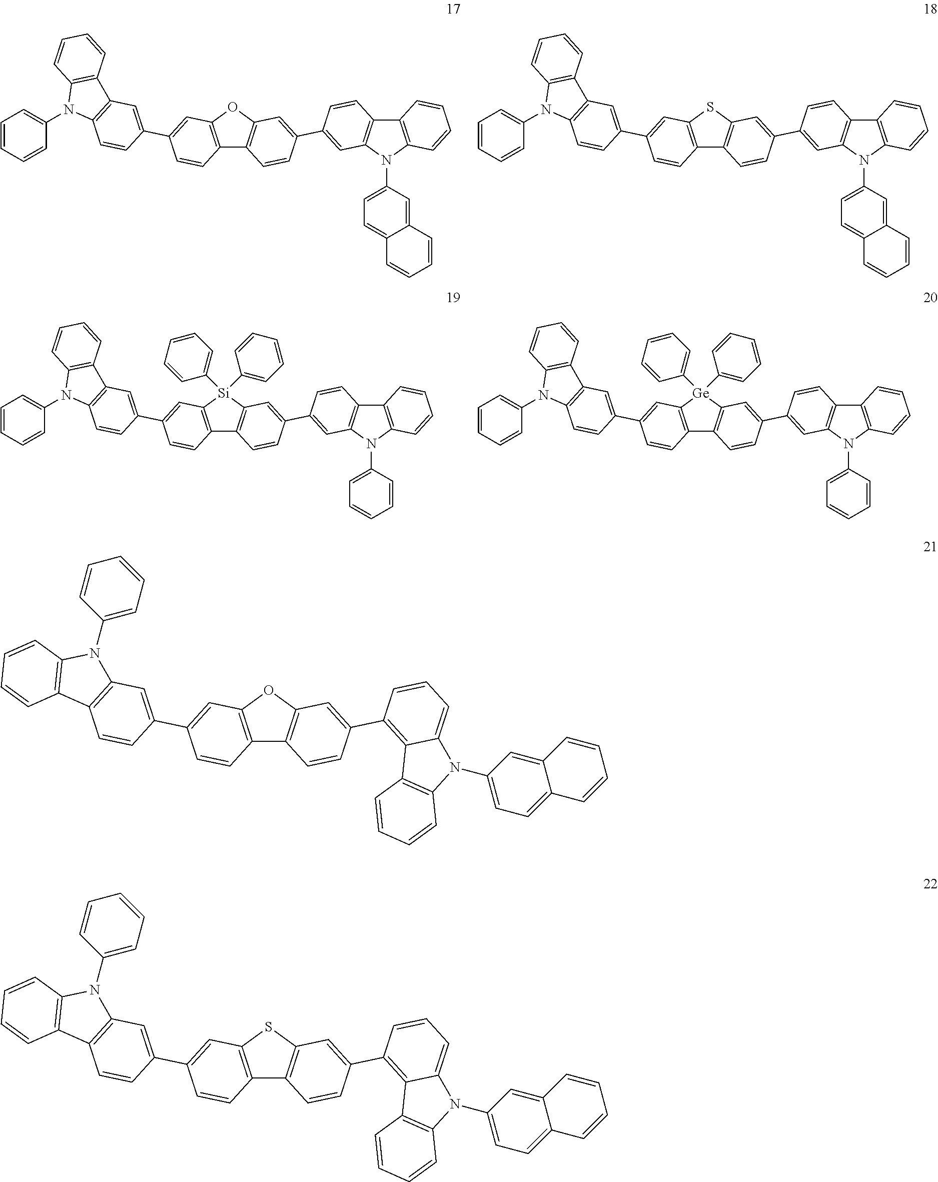

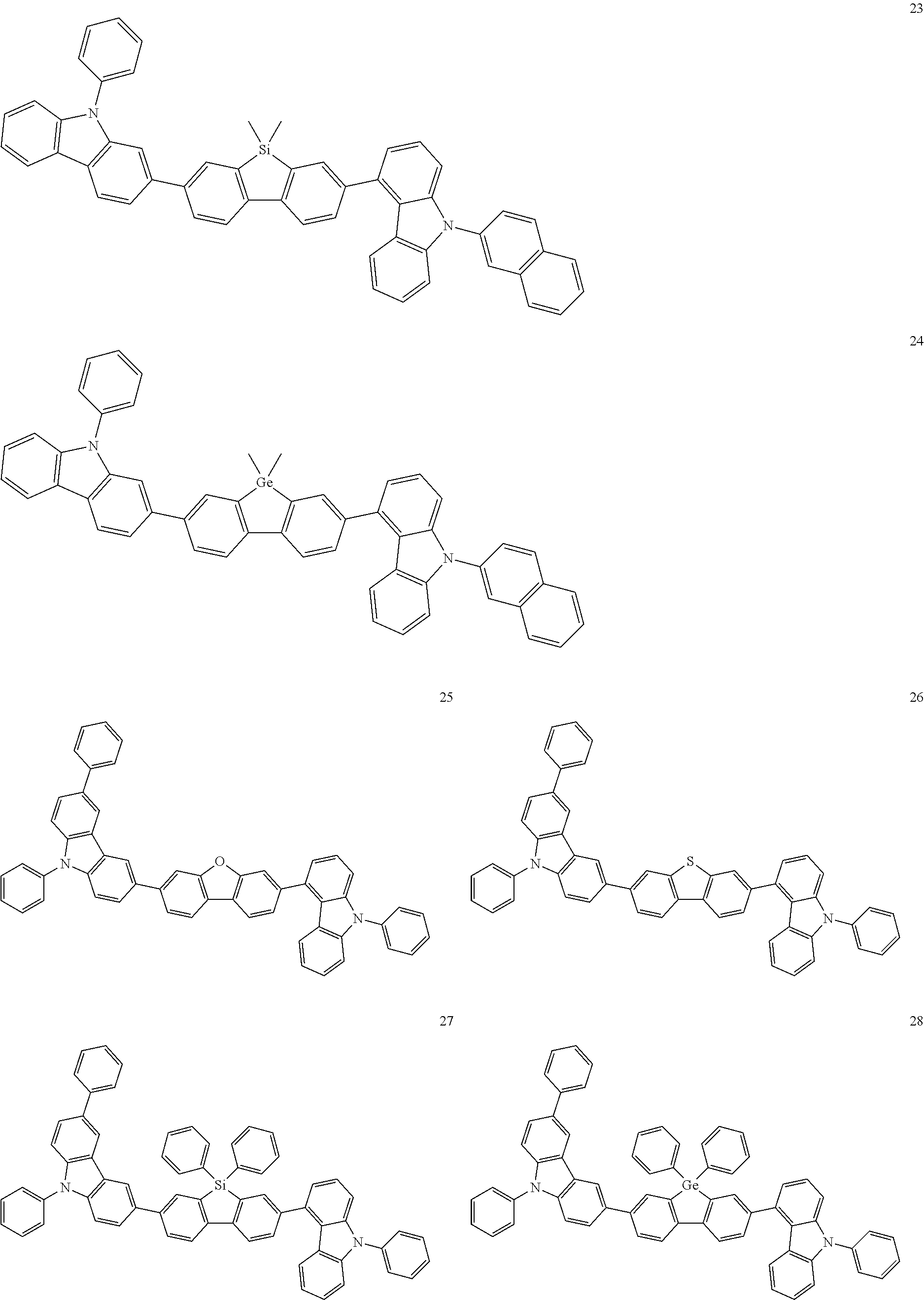

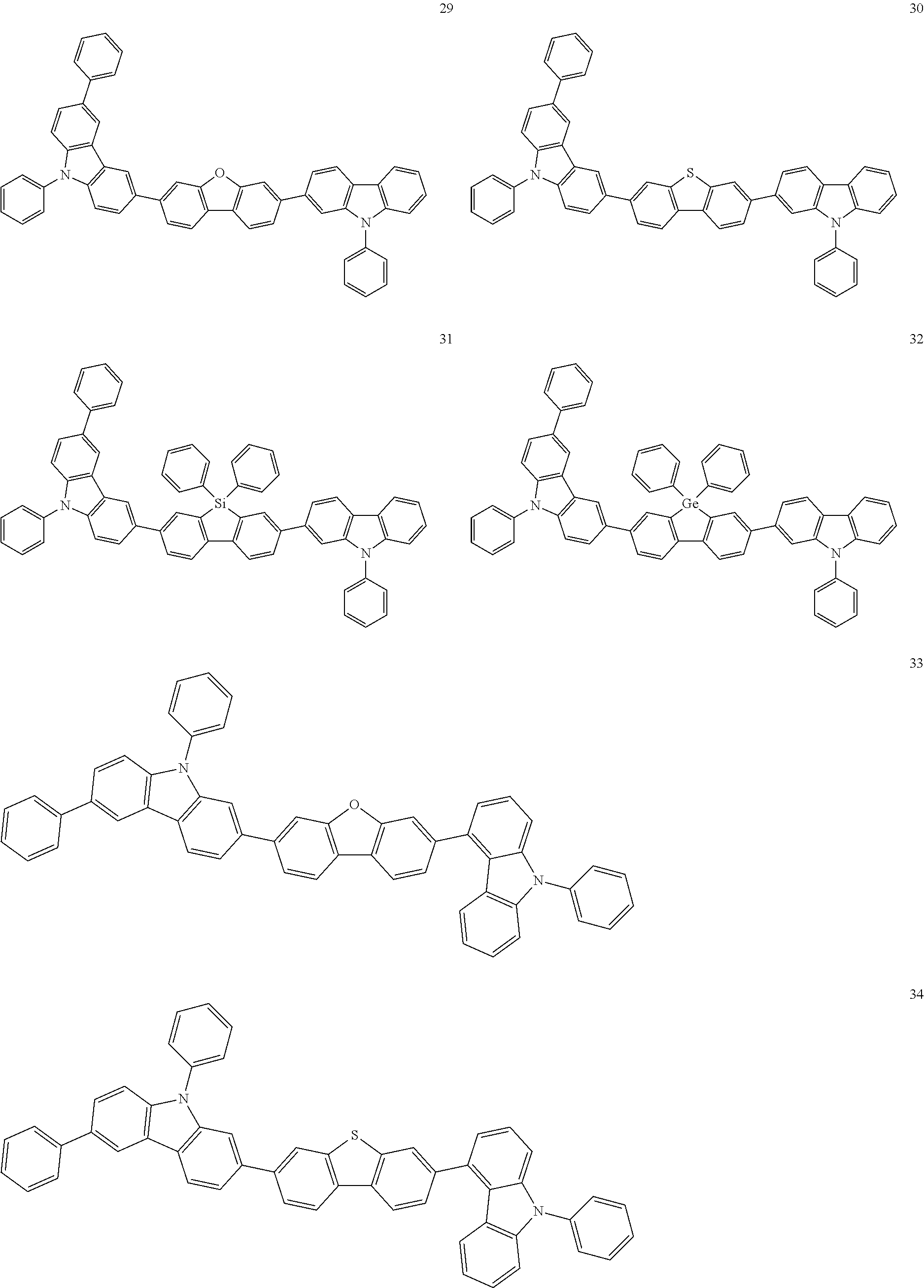

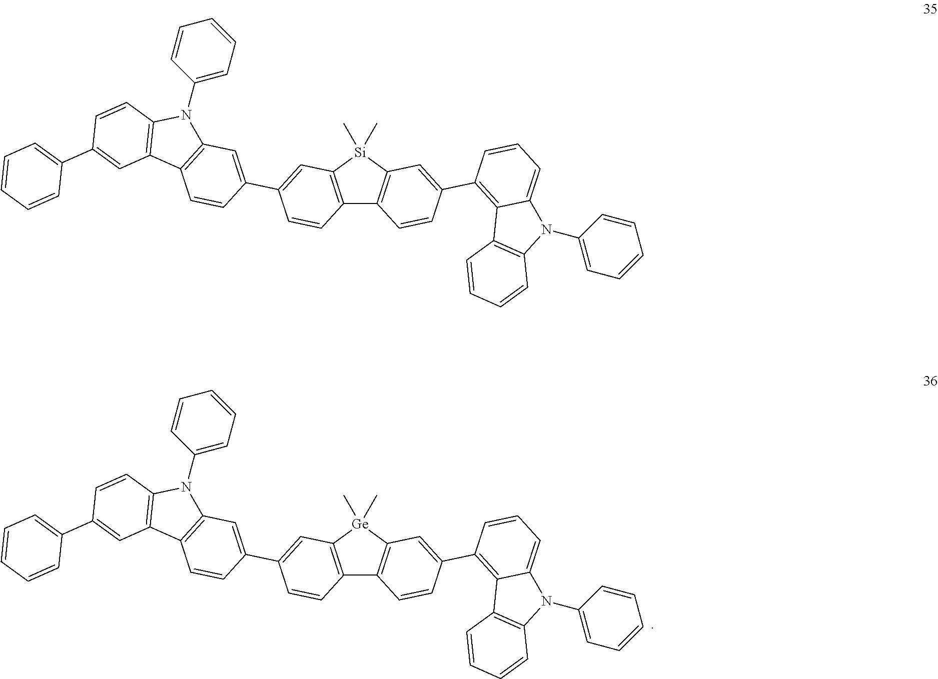

13. The organic EL device as claimed in claim 6, wherein the emission layer includes a condensed polycyclic aromatic compound.

14. The organic EL device as claimed in claim 13, wherein the condensed polycyclic aromatic compound is at least one compound selected from an anthracene derivative, a pyrene derivative, a fluoranthene derivative, a chrysene derivative, a benzanthracene derivative and a triphenylene derivative.

15. The organic EL device as claimed in claim 14, wherein the condensed polycyclic aromatic compound is at least one compound selected from a pyrene derivative and an anthracene derivative, the anthracene derivative being represented by the following Formula (2): 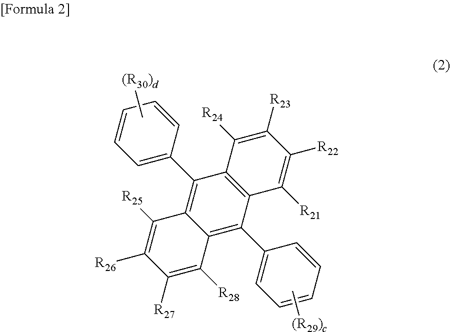
wherein, in the above Formula (2),
R₂₁ to R₃₀ are independently a substituent selected from hydrogen, deuterium, halogen, a substituted or unsubstituted silyl group, a substituted or unsubstituted C₁ to C₁₅ alkyl group, a substituted or unsubstituted C₆ to C₃₀ aryl group and a substituted or unsubstituted C₁ to C₃₀ heteroaryl group, or a substituted or unsubstituted aryl group or heteroaryl group formed by condensing at least two optional and adjacent R₂₁ to R₃₀, and c and d are independently an integer from 0 to 5.

16. The organic EL device as claimed in claim 13, wherein the condensed polycyclic aromatic compound includes at least one of the following compounds: 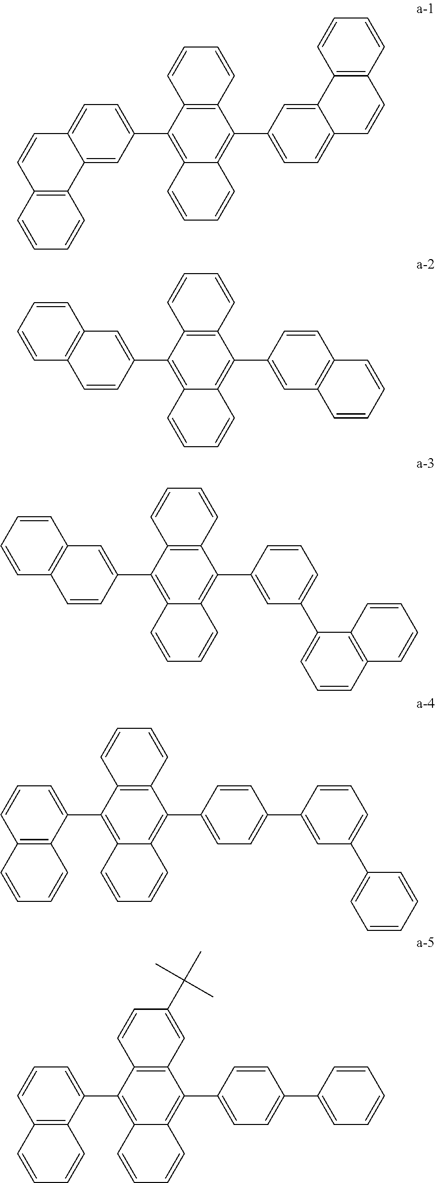 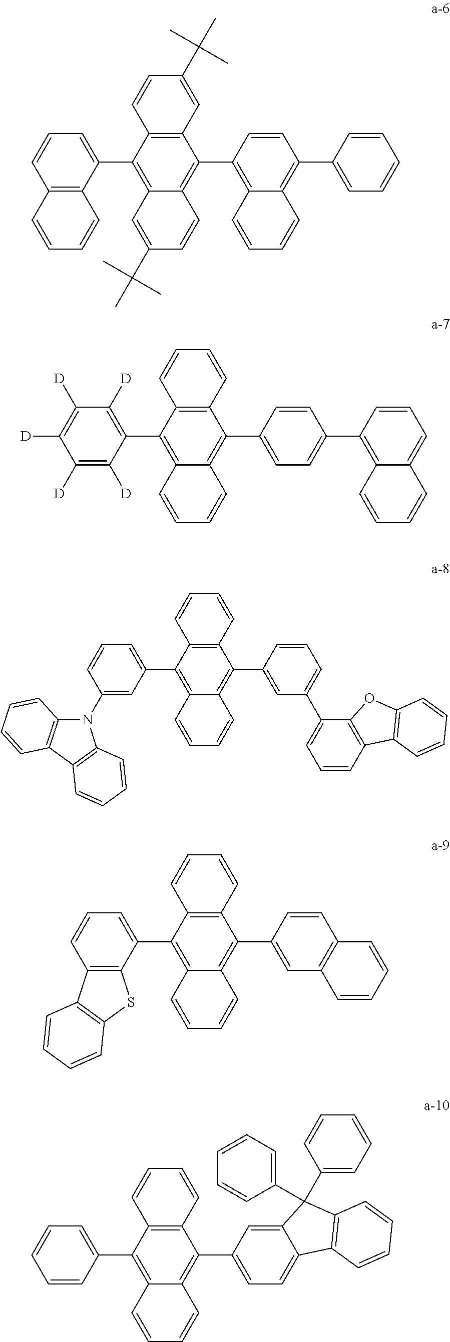 [chemical structure]
